(19) United States
(12) Patent Application Publication
(10) Pub. No.: US 2012/0269749 A1
(43) Pub. Date: Oct. 25, 2012

(54) COSMETIC COMPOSITIONS COMPRISING AN ESTER DERIVED FROM 4-CARBOXY-2-PYRROLIDINONE AND A MEROCYANIN SCREENING AGENT; USE OF SAID DERIVATIVE AS A SOLVENT FOR A MEROCYANIN SCREENING AGENT

(76) Inventors: Herve Richard, Gagny (FR); Benoit Muller, Paris (FR)

(21) Appl. No.: 13/508,199

(22) PCT Filed: Nov. 5, 2009
(86) PCT No.: PCT/EP2009/064696

§ 371 Date: Jul. 11, 2012

(51) Int. Cl. A61K 8/49; A61Q 17/04; C07D 207/28

(52) U.S. Cl. 424/59; 548/531

(57) ABSTRACT

The present invention relates to a composition comprising, in a cosmetically acceptable medium, at least one ester derived from 4-carboxy-2-pyrrolidinone, of formula (I) below:

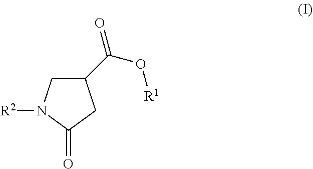

in which:
R¹ denotes a linear or branched C₁-C₂₀ alkyl radical,
R² denotes a linear or branched C₁-C₂₀ alkyl radical that may contain a C₅-C₆ ring, the phenyl radical, the benzyl radical or the phenethyl radical,
and at least one merocyanin-type UV screening agent.

The present invention also relates to the use of at least one ester derived from 4-carboxy-2-pyrrolidinone, of formula (I), as defined above, in a composition comprising, in a cosmetically acceptable medium, at least one merocyanin-type UV screening agent, as a solvent for said merocyanin screening agent, in and/or as an agent for improving the solubility of said merocyanin screening agent in said composition or with the aim of improving the sun protection factor.

The present invention relates to a composition comprising, in a cosmetically acceptable medium, at least one ester derived from 4-carboxy-2-pyrrolidinone, of formula (I), the definition of which will be given hereinafter, and at least one merocyanin UV-screening agent.

The present invention relates to the use of at least one ester derived from 4-carboxy-2-pyrrolidinone, of formula (I), the definition of which will be given hereinafter, in a composition comprising, in a cosmetically acceptable medium, at least one merocyanin UV-screening agent, as a solvent for said merocyanin screening agent and/or as an agent for improving the solubility of said active agent in said composition.

Many cosmetic or dermatological products are provided in various galenical forms comprising a liquid fatty phase, such as dispersions, oily lotions, oil/water, water/oil or multiple emulsions, or cream gels. Certain particularly advantageous cosmetic or dermatological active agents, such as lipophilic organic screening agents, are not readily soluble in the oily phase of these formulations and have a tendency during storage to form crystals or to precipitate, in particular in emulsions. Such phenomena are undesirable from the point of view of stability of the formulation and/or with respect to consumer comfort in so far as they can destabilise the composition and/or affect the aesthetic appearance of the product and/or lead to cosmetic discomfort upon application to the skin and/or the hair, or else to the concentration of active agents being limited in these formulations, which means that products that are sufficiently effective cannot be obtained.

In particular, anti-sun compositions are often in the form of an oil-in-water or water-in-oil emulsion, of gels or of anhydrous products which contain, at various concentrations, one or more lipophilic and/or hydrophilic, insoluble and/or soluble, organic and/or inorganic screening agents capable of selectively absorbing harmful UV radiation. These screening agents and the amounts thereof are selected according to the desired protection factor. Depending on whether they are lipophilic or, conversely, hydrophilic in nature, these screening agents can be distributed, respectively, either into the fatty phase or into the aqueous phase of the final composition.

UV screening agents of the merocyanin type are lipophilic long-UVA screening agents that are particularly advantageous for anti-sun cosmetic formulations. However, their photoprotective capacity when formulated is quite limited in the usual cosmetic carriers containing oils, such as oxyethylenated or oxypropylenated fatty (mono/poly)alcohols (“Cetiol HE” from Henkel or “Witconol APM” from Witco) or else fatty esters such as C₁₂-C₁₅ alkyl benzoate (“Finsolv TN” from Finetex), fatty acid triglycerides, for example Miglyol® 812 sold by the company Dynamit Nobel, or amino acid derivatives (“Eldew SL205” from Ajinomoto), because the solubility of these screening agents in these oils commonly used in formulation is not completely satisfactory. The result of this is: either the appearance over time of crystallisation in the formulations, which is detrimental to the good quality, stability and effectiveness of the anti-sun products; or the fact that the concentration of screening agents in the formulations has to be limited, thereby making it impossible to obtain sufficiently effective products.

There exists therefore the need to find novel solvents for effectively dissolving lipophilic screening agents of the merocyanin type in order to improve their solubility in the oils in the cosmetic or dermatological formulation carriers containing them, without the drawbacks listed above.

Pyrrolidone derivatives are known as penetrating agents for active agents such as oleocanthal in application WO 2008/01240 or such as asprotadil alkyl esters in U.S. Pat. No. 6,673,841.

However, the Applicant has now just discovered, surprisingly, a new family of effective solvents constituted of esters derived from 4-carboxy-2-pyrrolidinone, of formula (I), the definition of which will be given hereinafter, which make it possible to achieve this objective. These compounds can be incorporated into many cosmetic products.

This discovery forms the basis of the present invention.

The present invention relates in particular to a composition comprising, in a cosmetically acceptable medium, at least one ester derived from 4-carboxy-2-pyrrolidinone, of formula (I), and at least one merocyanin-type UV screening agent.

The present invention relates to the use of at least one ester derived from 4-carboxy-2-pyrrolidinone, of formula (I), the definition of which will be given hereinafter, in a composition comprising, in a cosmetically acceptable medium, at least one merocyanin-type UV screening agent, as a solvent for said active agent and/or as an agent for improving the solubility of said active agent in said composition.

The present invention also relates to the use of at least one ester derived from pyrrolidinone, of formula (I), in a composition comprising, in a cosmetically acceptable medium, at least one merocyanin-type UV screening agent, with the aim of improving the sun protection factor.

Other characteristics, aspects and advantages of the invention will become apparent upon reading the detailed description which follows.

The term “cosmetically acceptable” is intended to mean compatible with the skin and/or its appendages, which has a pleasant colour, odour and feel and which does not create any unacceptable discomfort (tingling, tautness, redness) that may dissuade the consumer from using this composition.

The esters derived from 4-carboxy-2-pyrrolidinone in accordance with the invention are chosen from those corresponding to general formula (I) below:

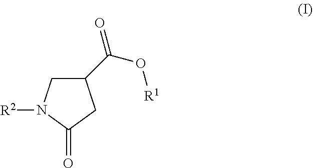

in which:
R¹ denotes a linear or branched C₁-C₂₀ alkyl radical,
R² denotes a linear or branched C₁-C₂₀ alkyl radical that may contain a C₅-C₆ ring, the phenyl radical, the benzyl radical or the phenethyl radical.

In formula (I), among the alkyl groups, mention may in particular be made of methyl, ethyl, n-propyl, isopropyl, n-butyl, isobutyl, tert-butyl, n-octyl, 2-ethylhexyl, dodecyl, hexadecyl, cyclohexyl or methylcyclohexyl groups.

Among the compounds of formula (I), use will more particularly be made of the following products (a) to (oo):

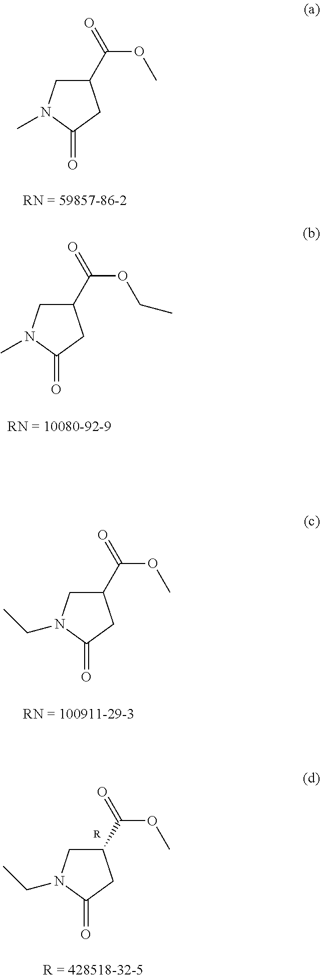

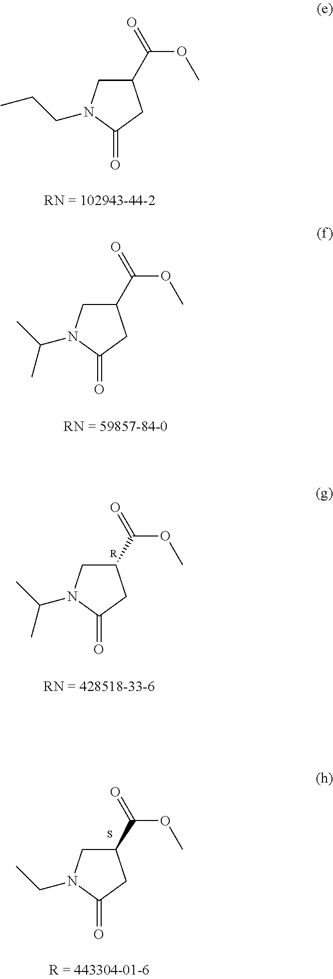

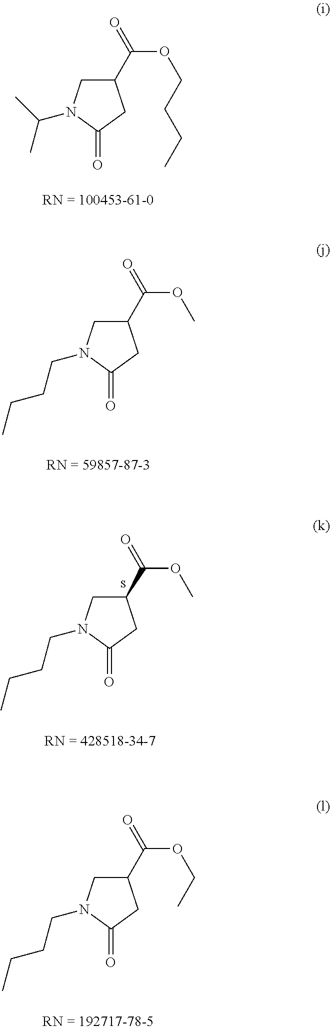

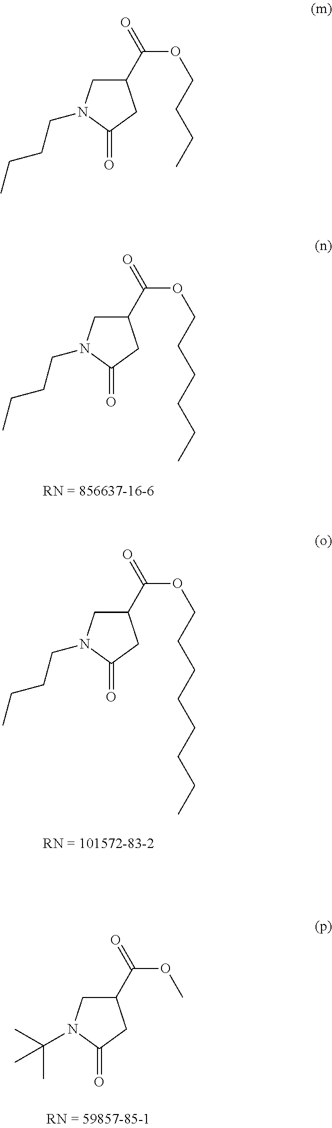

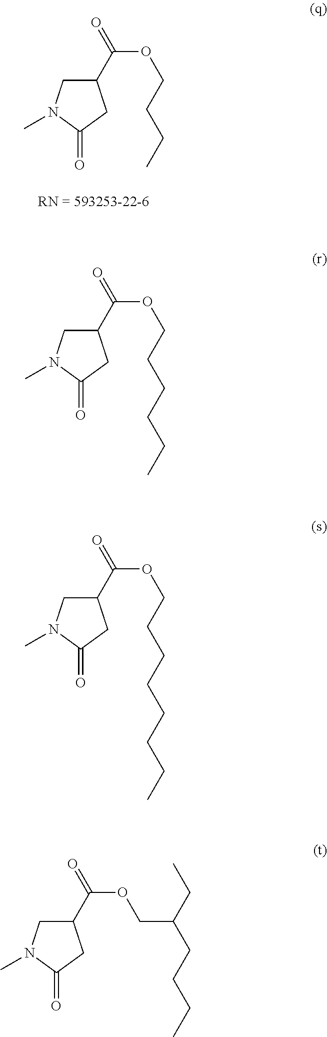

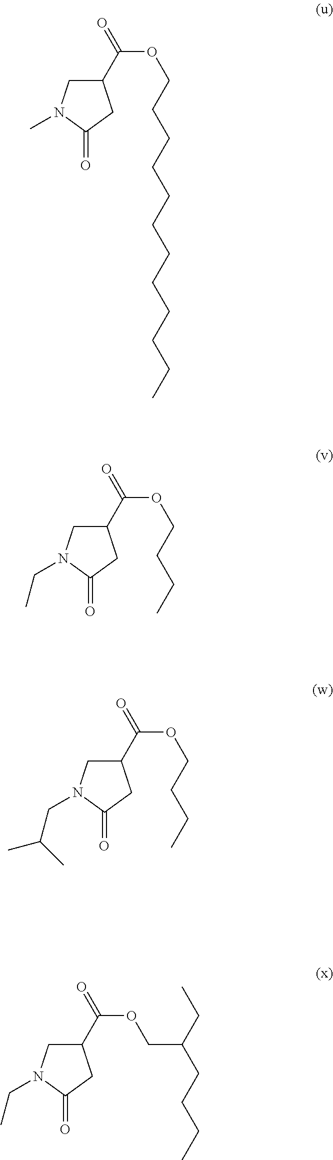

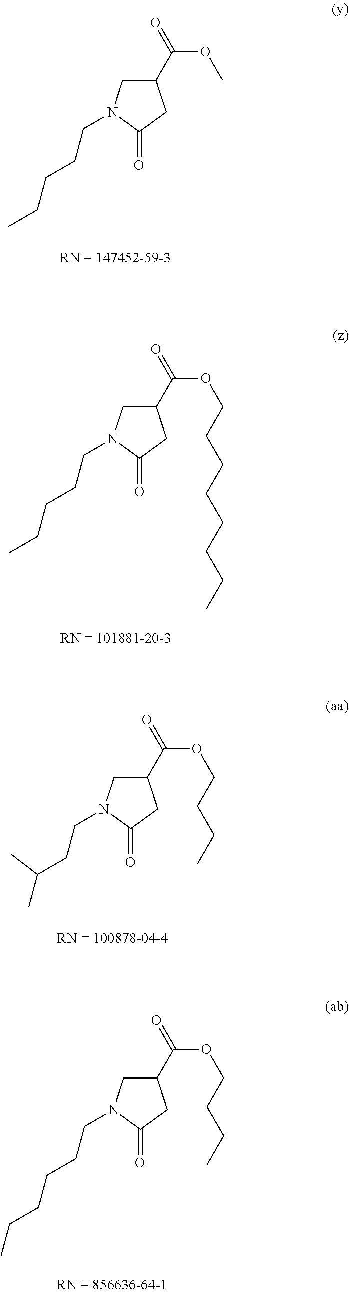

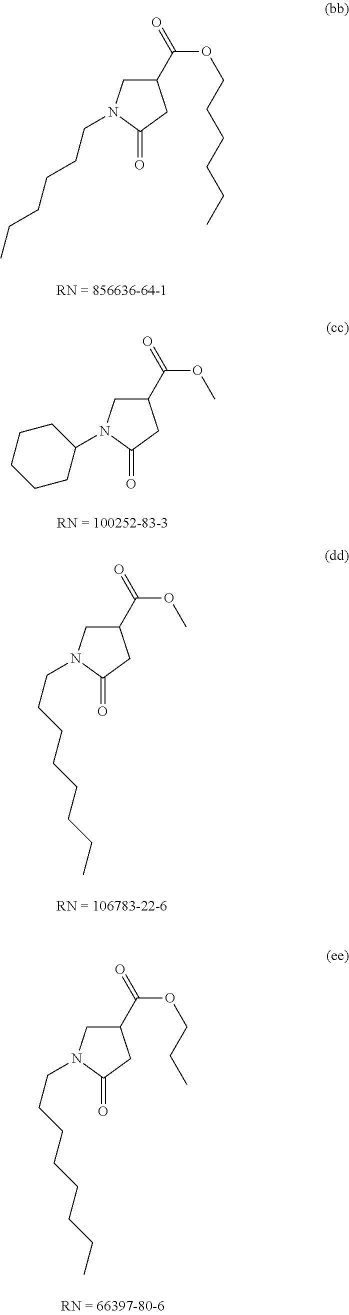

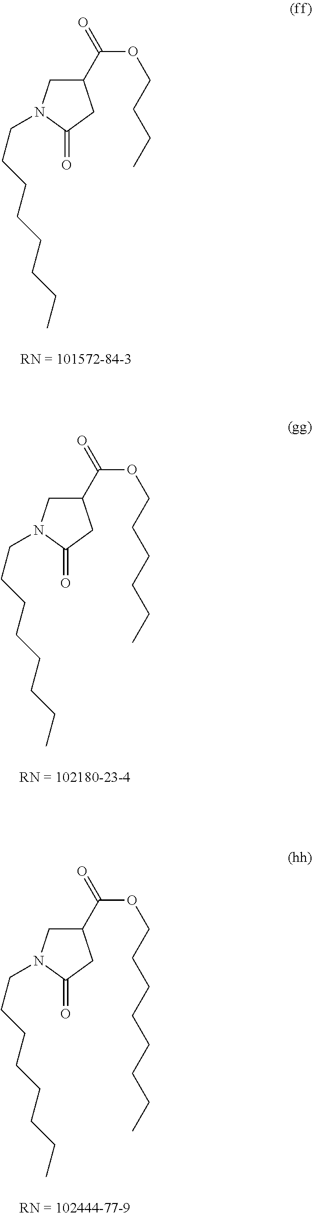

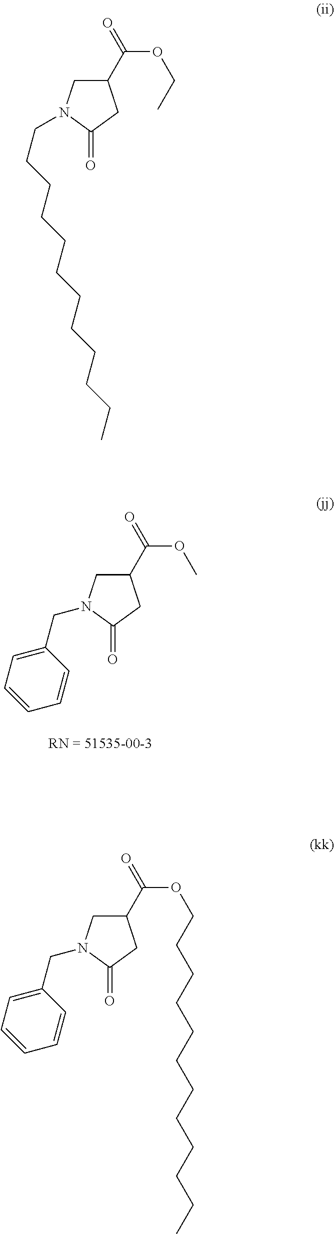

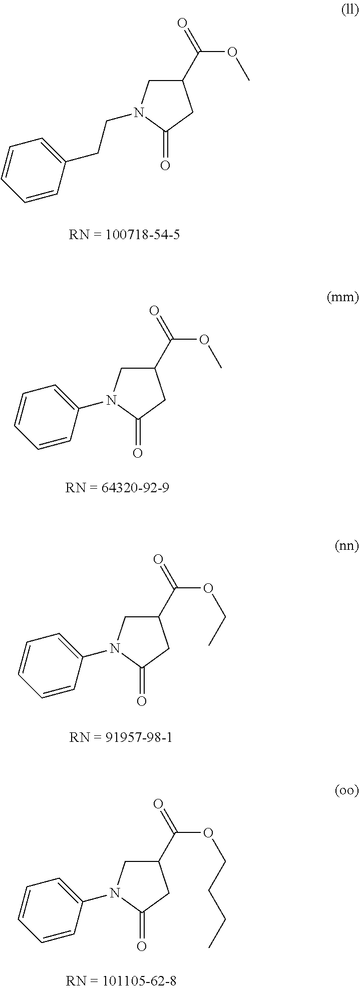

Use will more particularly be made of the derivatives of examples (t), (u), (v), (x), (j), (l), (m), (w), (n), (ab), (ii) and (kk), and even more particularly the compounds (j), (l) and (m).

The derivatives of formula (I), the syntheses of which are described in the following articles: J. Org. Chem., 26, pages 1519-24 (1961); Tetrahedron Asymmetric, 12 (23), pages 3241-9 (2001); J. Industrial & Engineering Chem., 47, pages 1572-8 (1955); J. Am. Chem. Soc., 60, pages 402-6 (1938); and in patents EP 0069512, U.S. Pat. No. 2,811,496 (1955), U.S. Pat. No. 2,826,588, U.S. Pat. No. 3,136,620, FR 2290199 and FR 2696744, can be readily obtained:
either by condensation of a diester of itaconic acid of formula (II) with a primary amine of formula (III), with or without solvent at a temperature between 20° C. and 150° C. according to the following scheme:

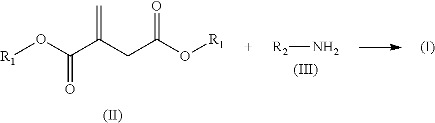

or in 2 stages starting from the itaconic acid of formula (IV) by condensation with the primary amine of formula (III), in the presence or absence of a solvent, so as to give the intermediate acid of formula (V), followed by esterification of this acid of formula (V) in the presence of an excess of alcohol of formula (VI) according to the following scheme:

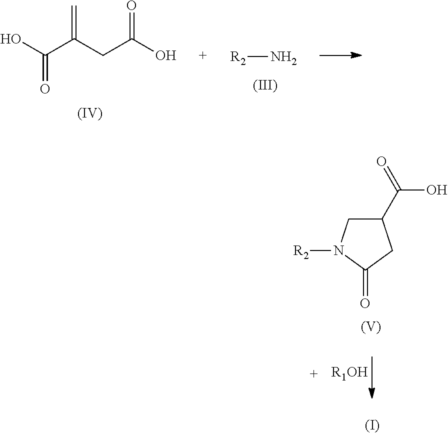

or the derivatives of formula (I) comprising an ester chain R′₁ (linear or branched C₃-C₂₀ alkyl radical) can also be obtained by transesterification of the derivatives comprising an ester chain R₁ (methyl or ethyl radical) in the presence of a linear or branched C₃-C₂₀ alcohol and of a tin catalyst or titanium catalyst according to the following scheme:

Preferably, the derivative(s) of formula (I) in accordance with the invention is(are) present in the compositions of the invention at contents ranging from 1% to 30% by weight, and more preferably from 3% to 20% by weight, relative to the total weight of the composition.

According to one particular embodiment of the invention, the derivative(s) of formula (I) in accordance with the invention is(are) present in an amount sufficient to solubilise, by itself or by themselves (without it being necessary to use another solvent) the entire amount of merocyanin(s) present in the composition.

The merocyanin-type UV screening agents that can be used may be from those described in applications

WO 2004/006878 and WO 2006/003094, the document IP COM Journal 4 (4), 16 No. IPCOM000011179D published on Apr. 3, 2004, and patent applications EP1965870, EP1962786 and EP1965869.

Among the merocyanin-type UV screening agents described in these documents, mention may be made of compounds (1) to (12) below:

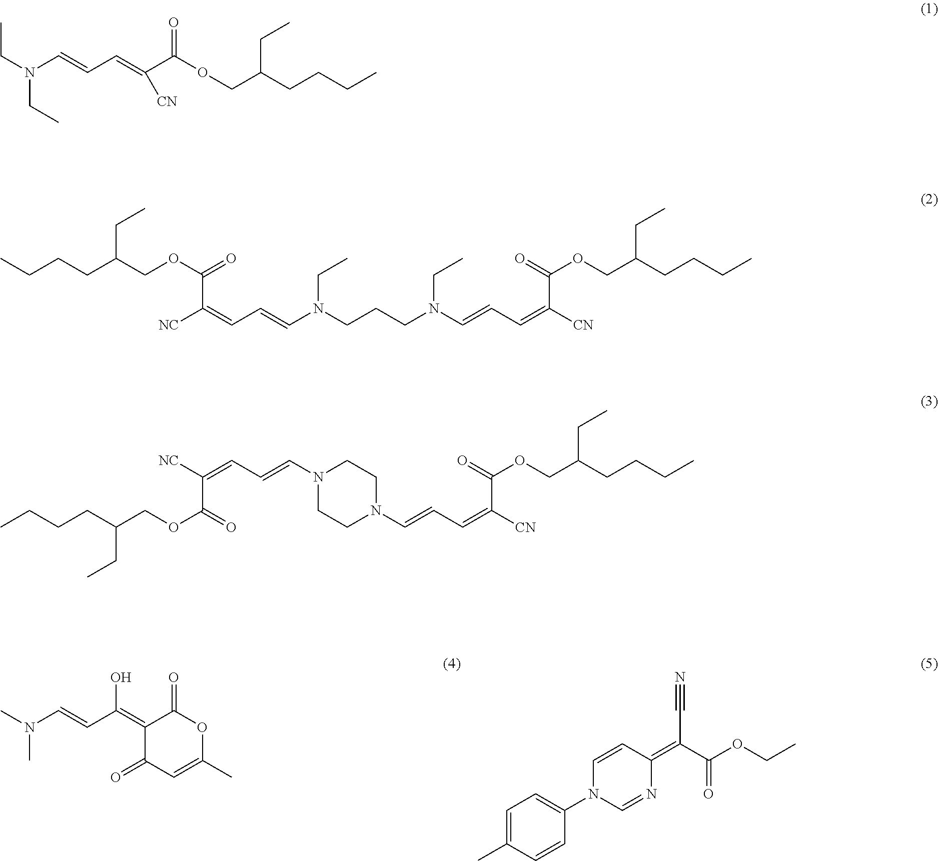

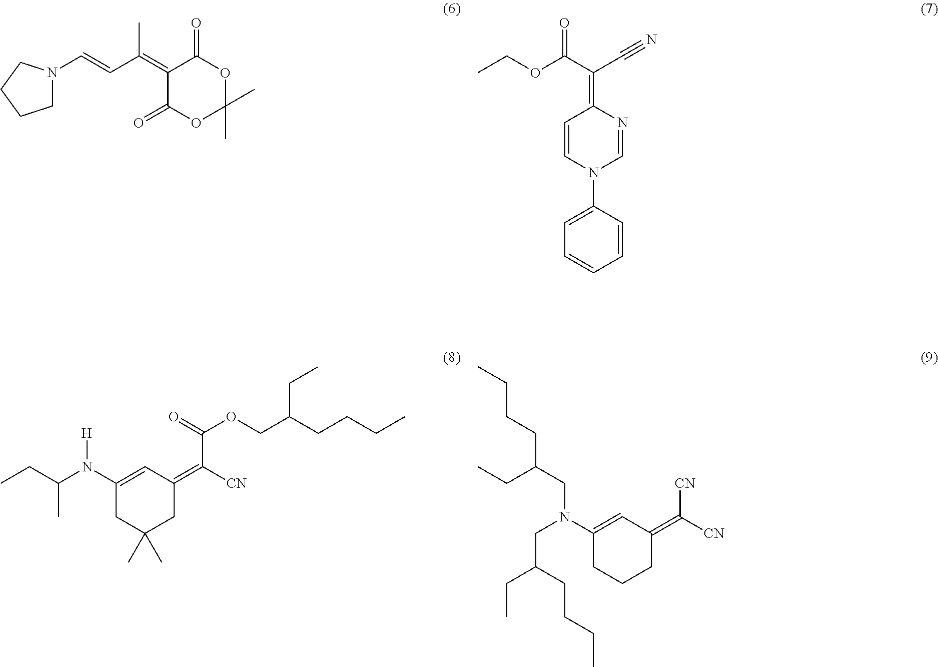

The compound {3-[(2-ethylhexyl)amino]-5,5-dimethyl-cyclohex-2-en-1-ylidene}propanedinitrile

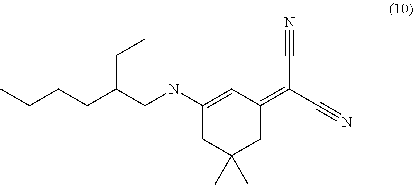

The compound {(2E)-3-[bis(2-ethylhexyl)amino]prop-2-en-1-ylidene}propanedinitrile

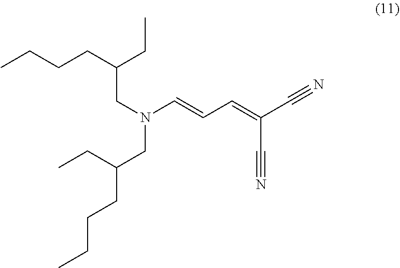

The compound 2-ethylhexyl (2E,4E)-2-cyano-5-(diethyl-amino)penta-2,4-dienoate

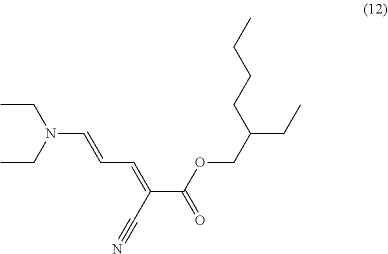

Among the merocyanin-type UV screening agents that can be used according to the invention, mention may more particularly be made of the sulphone derivates of merocyanin corresponding to one or other of formulae (VI) to (VIII) below:

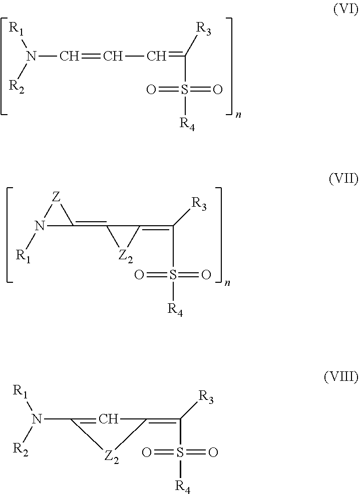

in which:
R₁ and R₂, which may be identical or different, represent H, a C₁-C₂₂ alkyl radical, a C₃-C₈ cycloalkyl radical or a C₆-C₂₀ aryl radical, in being understood that only one of R₁ and R₂ is H and that R₁ and R₂, together with the nitrogen, can form a ring containing the group —(CH₂)ₘ—, which is uninterrupted or interrupted with —O— or with —NH—,
R₃ represents a carboxyl, —COOR₅, —CONHR₅, —CONR₁R₅, —CN or SO₂R₅ group,
R₄ and R₅, which may be identical or different, represent a C₁-C₂₂ alkyl radical, a C₃-C₈ cycloalkyl radical or a C₆-C₂₀ aryl radical,
Z₁ and Z₂, which may be identical or different, are groups —(CH₂)₁—, uninterrupted or interrupted with —O—, with —S— or with —NR₆—, and/or unsubstituted or substituted with a C₁-C₆ alkyl radical,
R₆ is a C₁-C₅ alkyl radical,
l is 1-4,
m is 1-7,
n is 1-4,
   with the proviso that:
   (i) when n=2, R₁, R₄ or R₅ is an alkyl diradical or R₁ and R₂, together with 2 nitrogen atoms, form a —(CH₂)ₘ— divalent radical,
   (ii) when n=3, R₁, R₄ or R₅ is a trivalent radical,
   (iii) when n=4, R₁, R₄ or R₅ is a tetravalent radical,
   (iv) R₁ and R₂ are not simultaneously a hydrogen atom.

The compounds of formula (VI) may be in the E,E-, E,Z- or Z,Z-isomeric forms.

Among these three families of compounds, the compounds of formula (VI) are preferred.

The compounds of formula (VI) that are particularly preferred are those for which the following conditions are met:

R₁ and R₂, which may be identical or different, denote a C₁-C₁₂ alkyl;
R₃ denotes a COOR₅ group;
R₄ denotes a phenyl or tolyl group;
R₅ denotes a C₁-C₁₂ alkyl;
n is equal to 1 or 2.

By way of examples of compounds of formula (VI) that are particularly preferred, mention will be made of the products of formulae (13) to (17) below:
Ethyl 5-(dihexylamino)-2-(phenylsulphonyl)-2,4-penta-dienoate:

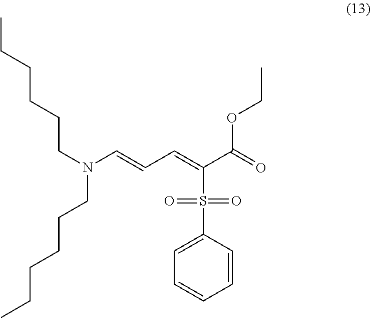

Octyl 5-N,N-diethylamino-2-phenylsulphonyl-2,4-penta-dienoate:

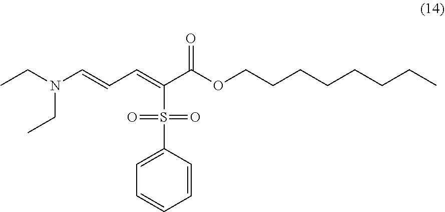

2-Ethylhexyl (2E,4E)-5-(diethylamino)-2-(phenyl-sulphonyl)penta-2,4-dienoate:

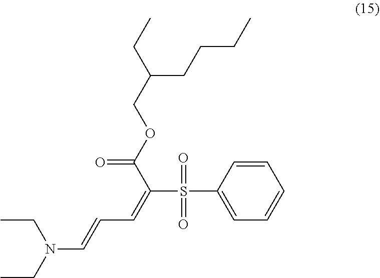

Lauryl 5-N,N-diethylamino-2-phenylsulphonyl-2,4-penta-dienoate:

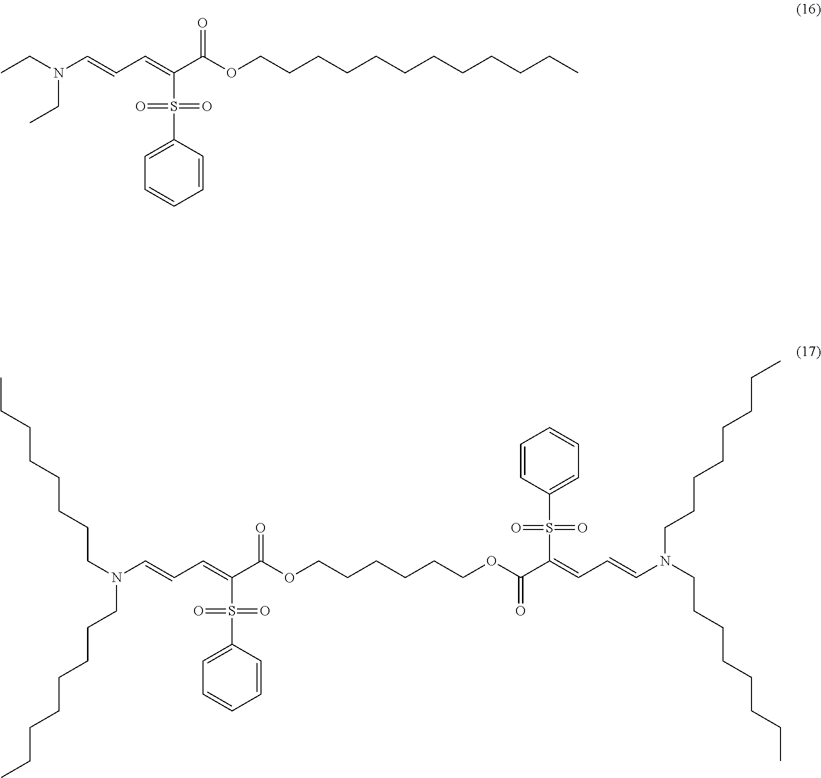

The syntheses of the derivatives of formulae (VI) to (VIII) are described in U.S. Pat. No. 2,186,608, U.S. Pat. No. 3,723,154, U.S. Pat. No. 4,045,229, U.S. Pat. No. 4,195,999, EP 0127819, EP 0210409, WO 2004/006878 and IPCOM000022279D.

Among the compounds that are sulphone derivatives of merocyanin in accordance with the present invention, mention may also be made of those corresponding to one or other of the following formulae (IX) to (XI):

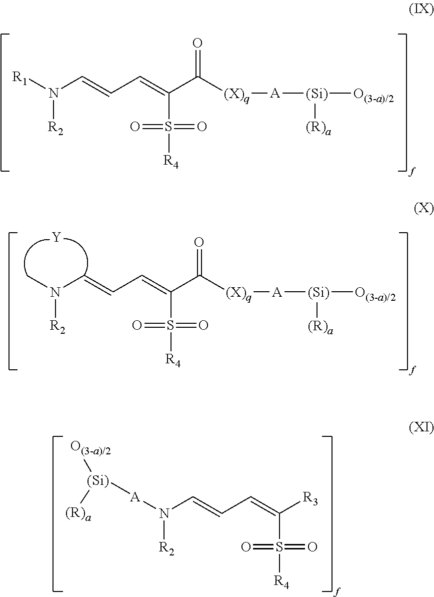

in which:
X represents —O— or —NR₅—,
   with R₁, R₂, R₃ and R₄ having the same definitions as in formulae (VI), (VII) and (VIII) indicated above,
o=0 or 1,
q=0 or 1,
Y is a divalent C₁-C₅ alkyl radical optionally substituted with C₁-C₄ alkyl radicals and/or containing —O—, —S— atoms, or with an —NR₁ group,
R, which may be identical or different, represent a linear or branched, and optionally halogenated, C₁-C₂₀ alkyl radical, a C₆-C₁₂ aryl radical or a C₁-C₁₀ alkoxy group,
a=0 to 3,
A is a divalent radical chosen from methylene, ethylene or a group corresponding to one of formulae (XII), (XIII) or (XIV) below:

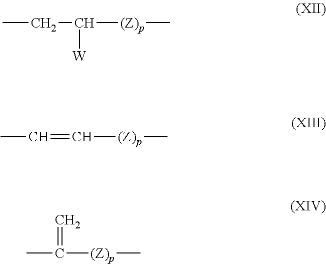

in which:
Z is a saturated or unsaturated, linear or branched C₁-C₆ alkylene radical optionally substituted with a hydroxyl radical or a saturated or unsaturated, linear or branched C₁-C₈ alkyl radical,
W represents a hydrogen atom, a hydroxyl radical or a saturated or unsaturated, linear or branched C₁-C₈ alkyl radical,
p is 0 or 1,
f=1 or 2.

The compounds of formulae (IX), (X) and (XI) may be present in the E,E-, E,Z- or Z,Z-isomeric forms.

In addition to the units of formula -A-(Si)(R)ₐ(O)_{(3-a)/2}, the organosiloxane may comprise units of formula (R)_{b}—(Si)(O)_{(4-b)/2} in which:

R has the same meaning as in formulae (VI) to (VIII), b=1, 2 or 3.

Preferably, the —(Si)(R)ₐ(O)_{(3-a)/2} groups may be represented by formula (XV), (XVI) or (XVII) below:

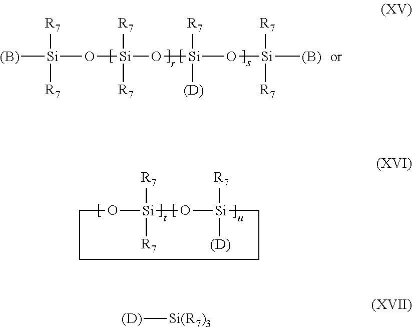

in which:
(D) is a linker between the silicone chain and the group A of the chromophores of formulae (IX) to (XI), R₇, may be identical or different, are chosen from linear or branched C₁-C₃₀ alkyl radicals and phenyl, 3,3,3-trifluoropropyl and trimethylsilyloxy radicals, at least 80% by number of the R₆ radicals being methyl,
(B), which may be identical or different, are chosen from the radicals R₈ and the radical A,
r is an integer between 0 and 200, limits included, and s is an integer between 0 and 50, limits included, and if s=0, at least one of the two symbols (B) denotes A,
u is an integer between 1 and 10, limits included, and t is an integer between 0 and 10, limits included, it being understood that t+u is greater than or equal to 3.

In formulae (VI) to (XI) above, the alkyl radicals may be linear or branched, saturated or unsaturated, and chosen in particular from methyl, ethyl, n-propyl, isopropyl, n-butyl, isobutyl, tert-butyl, n-amyl, isoamyl, neopentyl, n-hexyl, n-heptyl, n-octyl, 2-ethylhexyl and tert-octyl radicals. The particularly preferred alkyl radical is the methyl radical.

In formulae (VI) to (XI) above, the aryl radicals are preferably chosen from phenyl or tolyl.

Y is more particularly a group of atoms, the result of which is the formation of an oxazolidine ring, of a pyrrolidine ring, of a thiazolidine ring or of an indoline bicycle.

The linear or cyclic diorganosiloxanes of formula (XV) or (XVI) which fall within the context of the present invention are random polymers or oligomers preferably having at least one, and even more preferably all, of the following characteristics:
R₈ is preferably methyl,
B is preferably methyl (the case of the linear compounds of formula (XV)).

By way of examples of compounds of formula (IX) that are particularly preferred, mention will be made of the products of formulae (18) to (22) below:

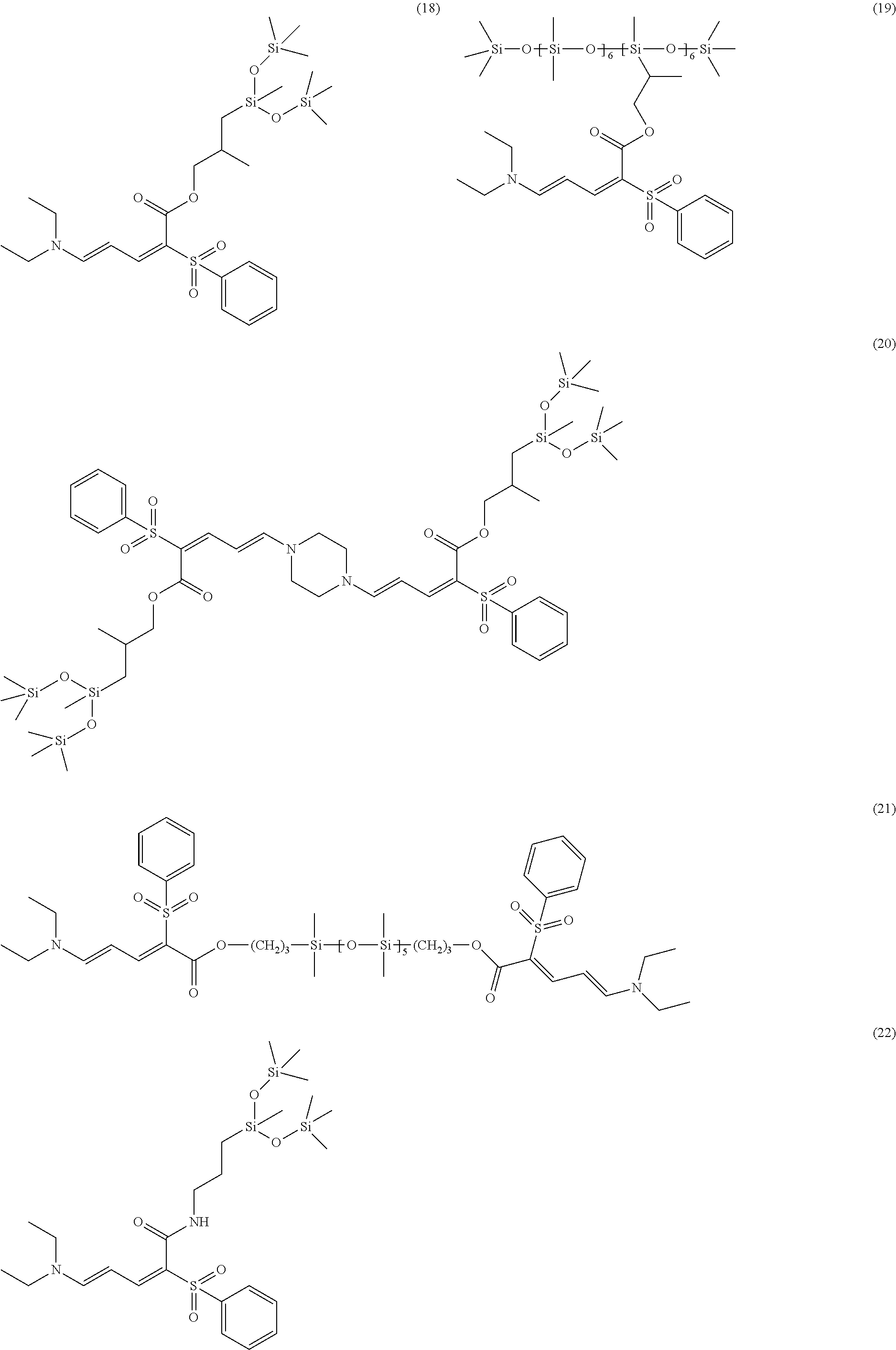

By way of examples of compounds of formula (X) that are particularly preferred, mention will be made of the mixture of compounds of formula (23) below:

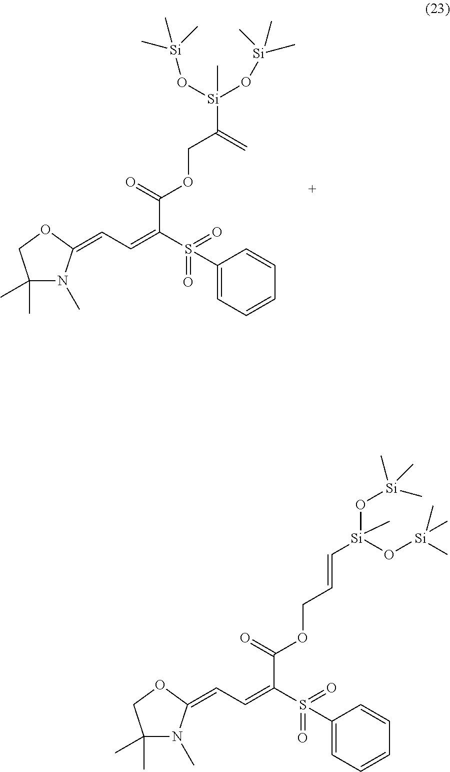

By way of examples of compounds of formula (XI) that are particularly preferred, mention will be made of the compounds of formula (24) to (29) below:

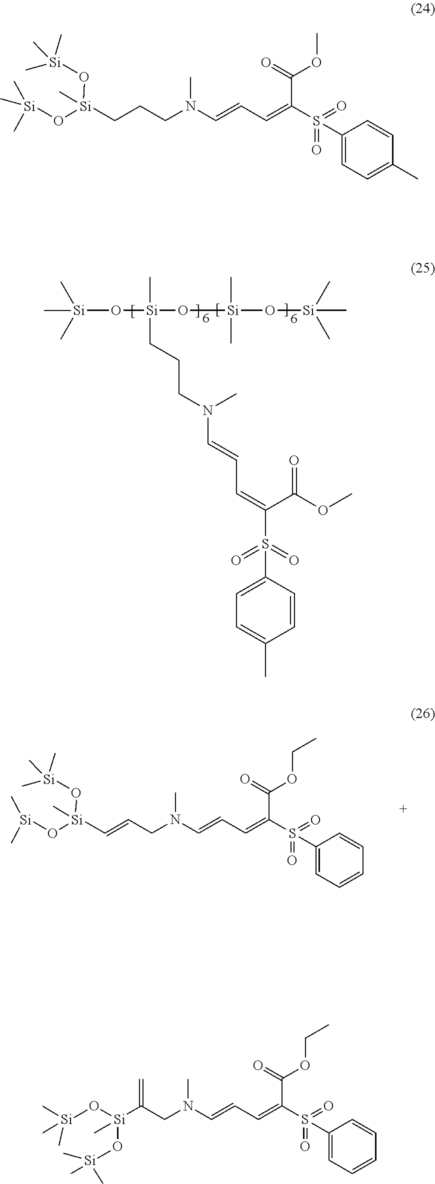

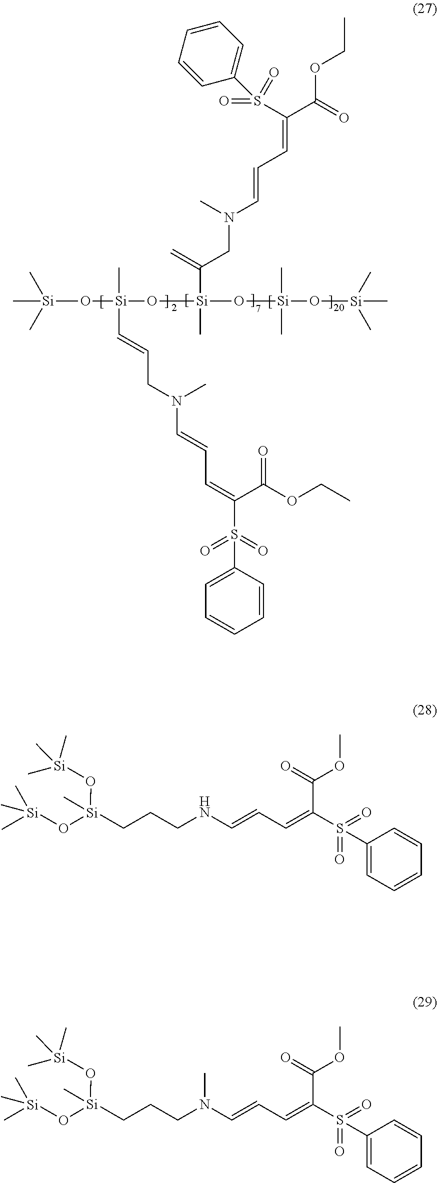

The merocyanin UV screening agents in accordance with the invention are preferably present in the compositions in accordance with the invention at contents of from 0.01% to 20% by weight, and more preferably from 0.1% to 10%, and even more preferably from 0.1% to 6% by weight, relative to the total weight of the composition.

The compositions of the invention may also contain other additional UV screening agents that are active in the UVA and/or UVB range.

The additional organic screening agents are in particular chosen from anthranilates; cinnamic derivatives; salicylic derivatives; camphor derivatives; benzophenone derivatives; β,β-dipheny-acrylate derivatives; triazine derivatives; benzotriazole derivatives; benzalmalonate derivatives, in particular those mentioned in U.S. Pat. No. 5,624,663; benzimidazole derivatives; imidazolines; bisbenzoazolyl derivatives as described in patents EP 669 323 and U.S. Pat. No. 2,463,264; p-aminobenzoic acid (PABA) derivatives; methylenebis(hydroxyphenylbenzotriazole) derivatives as described in applications U.S. Pat. No. 5,237,071, U.S. Pat. No. 5,166,355, GB 2303549, DE 197 26 184 and EP 893119; benzoxazole derivatives as described in patent applications EP 0832642, EP 1027883, EP 1300137 and DE 101 62 844; screening polymers and screening silicones such as those described in particular in application WO-93/04665; dimers derived from α-alkylstyrene, such as those described in patent application DE 198 55 649; 4,4-diarylbutadienes as described in applications EP 0967200, DE 197 46 654, DE 197 55 649, EP-A-1008586, EP 1133980 and EP 133981, and mixtures thereof.

As examples of additional organic photoprotective agents, mention may be made of those denoted below under their INCI name:

### Dibenzoylmethane Derivatives:

Butyl methoxydibenzoylmethane sold in particular under the trade name “PARSOL 1789” by Hoffmann Laroche,
Isopropyl dibenzoylmethane.

### Cinnamic Derivatives:

Ethylhexyl methoxycinnamate sold in particular under the trade name Parsol MCX by DSM Nutritional Products, Inc.,
Isopropyl methoxycinnamate,
Isoamyl methoxycinnamate sold under the trade name Neo Heliopan E 1000 by Symrise,
DEA methoxycinnamate,
Diisopropyl methylcinnamate,
Glyceryl ethylhexanoate dimethoxycinnamate.

### Para-Aminobenzoic Acid Derivatives:

### PABA,

### Ethyl PABA,

Ethyl dihydroxypropyl PABA,
Ethylhexyl dimethyl PABA sold in particular under the name “Escalol 507” by ISP,

### Glyceryl PABA,

PEG-25 PABA sold under the name “Uvinul P25” by BASF.

### Salicylic Derivatives:

Homosalate sold under the name “Eusolex HMS” by Rona/EM Industries,
Ethylhexyl salicylate sold under the name “Neo Heliopan OS” by Symrise,
Dipropylene glycol salicylate sold under the name “Dipsal” by Scher,
TEA salicylate, sold under the name “Neo Heliopan TS” by Symrise.

### β,β-Diphenylacrylate Derivatives:

Octocrylene sold in particular under the trade name “Uvinul N539” by BASF,
Etocrylene, sold in particular under the trade name “Uvinul N35” by BASF.

### Benzophenone Derivatives:

Benzophenone-1 sold under the trade name “Uvinul 400” by BASF,
Benzophenone-2 sold under the trade name “Uvinul D50” by BASF,
Benzophenone-3 or oxybenzone, sold under the trade name “Uvinul M40” by BASF,
Benzophenone-4 sold under the trade name “Uvinul MS40” by BASF,

### Benzophenone-5,

Benzophenone-6 sold under the trade name “Helisorb 11” by Norquay,
Benzophenone-8 sold under the trade name “Spectra-Sorb UV-24” by American Cyanamid,
Benzophenone-9 sold under the trade name “Uvinul DS-49” by BASF,

### Benzophenone-12,

n-Hexyl 2-(4-diethylamino-2-hydroxybenzoyl)benzoate sold under the trade name “Uvinul A+” by BASF.

### Benzylidenecamphor Derivatives:

3-Benzylidenecamphor manufactured under the name “Mexoryl SD” by Chimex,
4-Methylbenzylidenecamphor sold under the name “EUSOLEX 6300” by Merck,
Benzylidenecamphorsulphonic acid manufactured under the name “Mexoryl SL” by Chimex,
Camphor benzalkonium methosulphate manufactured under the name “Mexoryl SO” by Chimex,
Terephthalylidenedicamphorsulphonic acid manufactured under the name “Mexoryl SX” by Chimex,
Polyacrylamidomethylbenzylidenecamphor manufactured under the name “Mexoryl SW” by Chimex.

### Phenylbenzimidazole Derivatives:

Phenylbenzimidazolesulphonic acid sold in particular under the trade name “EUSOLEX 232” by Merck,
Disodium phenyl dibenzimidazole tetrasulphonate sold under the trade name “Neo Heliopan AP” by Symrise.

### Phenylbenzotriazole Derivatives:

Drometrizole trisiloxane sold under the name “Silatrizole” by Rhodia Chimie,
Methylenebis(benzotriazolyl)tetramethylbutylphenol, sold in solid form under the trade name “Mixxim BB/100” by Fairmount Chemical, or in micronised form as an aqueous dispersion under the trade name “Tinosorb M” by CIBA Specialty Chemicals.

### Triazine Derivatives:

Bis-ethylhexyloxyphenol methoxyphenyl triazine sold under the trade name “Tinosorb S” by Ciba Geigy,
Ethylhexyl triazone sold in particular under the trade name “Uvinul T150” by BASF,
Diethylhexyl butamido triazone sold under the trade name “Uvasorb HEB” by Sigma 3V,
2,4,6-tris(dineopentyl 4′-aminobenzalmalonate)-s-triazine,
2,4,6-tris(diisobutyl 4′-aminobenzalmalonate)-s-triazine,
2,4-bis(dineopentyl 4′-aminobenzalmalonate)-6-(n-butyl 4′-aminobenzoate)-s-triazine,
the symmetrical triazine screening agents described in U.S. Pat. No. 6,225,467, application WO 2004/085412 (see compounds 6 and 9) or the document “Symetrical Triazine Derivatives” IP.COM Journal, IP.COM INC West Henrietta, NY, US (20 Sep. 2004), especially 2,4,6-tris(biphenyl)-1,3,5-triazines (in particular 2,4,6-tris(biphenyl)-4-yl-1,3,5-triazine) and 2,4,6-tris(terphenyl)-1,3,5-triazine which is also mentioned in patent applications WO 06/035000, WO 06/034982, WO 06/034991, WO 06/035007, WO 2006/034992 and WO 2006/034985.

### Anthranilic Derivatives:

Menthyl anthranilate sold under the trade name “Neo Heliopan MA” by Haarmann and Reimer.

### Imidazoline Derivatives:

Ethylhexyldimethoxybenzylidenedioxoimidazoline propionate.

### Benzalmalonate Derivatives:

Polyorganosiloxane containing benzalmalonate functions, for instance Polysilicone-15, sold under the trade name “Parsol SLX” by DSM Nutritional Products, Inc.

### 4,4-Diarylbutadiene Derivatives:

1,1-Dicarboxy(2,2′-dimethylpropyl)-4,4-diphenylbutadiene.

### Benzoxazole Derivatives:

2,4-bis[5-1-(dimethylpropyl)benzoxazol-2-yl(4-phenyl)-imino]-6-(2-ethylhexyl)imino-1,3,5-triazine sold under the name Uvasorb K2A by Sigma 3V,
and mixtures thereof.

The preferred organic screening agents are chosen from:

Butyl methoxydibenzoylmethane,
Ethylhexyl methoxycinnamate,
Ethylhexyl salicylate,

### Homosalate,

### Octocrylene,

Phenylbenzimidazolesulphonic acid,

### Benzophenone-3,

n-Hexyl 2-(4-diethylamino-2-hydroxybenzoyl)benzoate,
Terephthalylidenedicamphorsulphonic acid,
Disodium phenyl dibenzimidazole tetrasulphonate,

### Methylenebis(benzotriazolyl)tetramethylbutylphenol,

Bis-ethylhexyloxyphenol methoxyphenyl triazine,
Ethylhexyl triazone,
Diethylhexyl butamido triazone,
2,4,6-tris(dineopentyl 4′-aminobenzalmalonate)-s-triazine,
2,4,6-tris(diisobutyl 4′-aminobenzalmalonate)-s-triazine,
2,4-bis(dineopentyl 4′-aminobenzalmalonate)-6-(n-butyl4′-aminobenzoate)-s-triazine,
2,4,6-tris(Biphenyl)-4-yl-1,3,5-triazine,
Drometrizole trisiloxane,

### Polysilicone-15,

and mixtures thereof.

Of course, those skilled in the art will take care to select the optional additional screening agent(s) and/or the amounts thereof in such a way that the advantageous properties intrinsically associated with the compositions in accordance with the invention are not, or not substantially, impaired by the envisaged addition(s).

The additional organic screening agents in accordance with the invention represent, in general, from 0.1% to 30%, preferably from 1% to 25%, of the total weight of the composition.

The compositions according to the invention may also contain artificial skin tanning and/or browning agents (self-tanning agents), and more particularly dihydroxyacetone (DHA). They are preferably present in amounts ranging from 0.1% to 10% by weight relative to the total weight of the composition.

The aqueous compositions in accordance with the present invention may also comprise conventional cosmetic adjuvants chosen in particular from fatty substances, organic solvents, ionic or non-ionic, hydrophilic or lipophilic thickeners, demulcents, humectants, opacifiers, stabilisers, emollients, silicones, antifoams, fragrances, preservatives, anionic, cationic, non-ionic, zwitterionic or amphoteric surfactants, active agents, fillers, polymers, propellants and basifying or acidifying agents, or any other ingredient normally used in the cosmetics and/or dermatological field.

Of course, those skilled in the art will take care to select the optional additive(s) and/or the amounts thereof in such a way that the advantageous properties intrinsically associated with the compositions in accordance with the invention are not, or not substantially, impaired by the envisaged addition(s). The fatty substances may be constituted of an oil or a wax other than the apolar waxes as defined previously or mixtures thereof. The term “oil” is intended to mean a compound that is liquid at ambient temperature. The term “wax” is intended to mean a compound that is solid or substantially solid at ambient temperature, and the melting point of which is generally above 35° C.

As oils, mention may be made of mineral oils (paraffin); plant oils (sweet almond oil, macadamia oil, blackcurrant seed oil, jojoba oil); synthetic oils such as perhydrosqualene, alcohols, fatty amides (such as isopropyl lauroyl sarcosinate sold under the name “Eldew SL-205” by the company Ajinomoto), fatty acids or esters such as the C₁₂-C₁₅ alkyl benzoate sold under the trade name “Finsolv TN” or “Witconol TN” by the company Witco, 2-ethylphenyl benzoate such as the commercial product sold under the name X-Tend 226® by the company ISP, octyl palmitate, isopropyl lanolate, triglycerides, including those of capric/caprylic acid, dicaprylyl carbonate sold under the name “Cetiol CC” by the company Cognis, oxyethylenated or oxypropylenated fatty esters and ethers; silicone oils (cyclomethicone, polydimethylsiloxanes or PDMSs) or fluoro oils, and polyalkylenes.

As waxy compounds, mention may be made of carnauba wax, beeswax, hydrogenated castor oil, polyethylene waxes and polymethylene waxes, such as that sold under the name Cirebelle 303 by the company Sasol.

Among the organic solvents, mention may be made of lower alcohols and polyols. The latter may be chosen from glycols and glycol ethers such as ethylene glycol, propylene glycol, butylene glycol, dipropylene glycol or diethylene glycol.

As hydrophilic thickeners, mention may be made of carboxyvinyl polymers such as Carbopols (Carbomers) and Pemulens (acrylate/C₁₀-C₃₀ alkyl acrylate copolymer); polyacrylamides such as, for example, the crosslinked copolymers sold under the names Sepigel 305 (C.T.F.A. name: polyacrylamide/C₁₃₋₁₄ isoparaffin/Laureth 7) or Simulgel 600 (C.T.F.A. name: acrylamide/sodium acryloyldimethyltaurate copolymer/isohexadecane/polysorbate 80) by the company Seppic; polymers and copolymers of 2-acrylamido-2-methylpropanesulphonic acid, optionally crosslinked and/or neutralised, such as the poly(2-acrylamido-2-methylpropanesulphonic acid) sold by the company Hoechst under the trade name “Hostacerin AMPS” (CTFA name: ammonium polyacryloyldimethyl taurate) or Simulgel 800 sold by the company Seppic (CTFA name: sodium polyacryolyldimethyl taurate/polysorbate 80/sorbitan oleate); copolymers of 2-acrylamido-2-methylpropanesulphonic acid and of hydroxyethyl acrylate, such as Simulgel NS and Sepinov EMT 10 sold by the company Seppic; cellulose derivatives such as hydroxyethylcellulose; polysaccharides and especially gums such as xanthan gum; and mixtures thereof.

As lipophilic thickeners, mention may be made of synthetic polymers such as poly(C₁₀-C₃₀ alkyl acrylates) sold under the name “Intelimer IPA 13-1” and “Intelimer IPA 13-6” by the company Landec, or else modified clays such as hectorite and derivatives thereof, for instance the products sold under the name Bentone.

Among the active agents, mention may be made of:
vitamins (C, K, PP, etc.) and derivatives or precursors thereof, alone or as mixtures;
agents for combating pollution and/or free-radical scavengers;
depigmenting agents and/or propigmenting agents;
anti-glycation agents;
calmatives;
NO-synthase inhibitors;
agents for stimulating the synthesis of dermal or epidermal macromolecules and/or for preventing their degradation;
agents for stimulating fibroblast proliferation;
agents for stimulating keratinocyte proliferation;
myorelaxants;
tensioning agents;
matting agents;
keratolytic agents;
desquamating agents;
moisturisers;
anti-inflammatory agents;
agents that act on the energy metabolism of cells;
insect repellents;
substance P antagonists and CRGP antagonists;
agents for preventing hair loss and/or for hair regrowth; and
anti-wrinkle agents.

Of course, those skilled in the art will take care to select the optional additional compound(s) mentioned above and/or the amounts thereof in such a way that the advantageous properties intrinsically associated with the compositions in accordance with the invention are not, or are not substantially, impaired by the envisaged addition(s).

The compositions according to the invention can be prepared according to techniques well known to those skilled in the art. They may in particular be in the form of a simple or complex emulsion (O/W, W/O, O/W/O or W/O/W) such as a cream, a milk or a cream gel; in the form of an aqueous gel; or in the form of a lotion. They may optionally be packed in an aerosol and be in the form of a foam or a spray.

Preferably, the compositions according to the invention are in the form of an oil-in-water or water-in-oil emulsion.

The emulsions generally contain at least one emulsifier chosen from amphoteric, anionic, cationic or non-ionic emulsifiers, used alone or as a mixture. The emulsifiers are chosen in an appropriate manner according to the emulsion to be obtained (W/O or O/W). The emulsions may also contain other types of stabilisers, for instance fillers or gelling or thickening polymers.

As emulsifying surfactants that can be used for preparing W/O emulsions, mention may be made, for example, of alkyl esters or ethers of sorbitan, of glycerol or of sugars; silicone surfactants, for instance dimethicone copolyols, such as the mixture of cyclomethicone and of dimethicone copolyol sold under the name “DC 5225 C” by the company Dow Corning, and alkyl dimethicone copolyols, such as the lauryl methicone copolyol sold under the name “Dow Corning 5200 Formulation Aid” by the company Dow Corning; cetyl dimethicone copolyol, such as the product sold under the name Abil EM 90R by the company Goldschmidt and the mixture of cetyl dimethicone copolyol, of polyglycerol isostearate (4 mol) and of hexyl laurate sold under the name Abil WE 09 by the company Goldschmidt. It is possible to also add thereto one or more coemulsifiers, which advantageously may be chosen from the group comprising alkylated polyol esters.

As alkylated polyol esters, mention may in particular be made of polyethylene glycol esters, for instance PEG-30 dipolyhydroxystearate, such as the product sold under the name Arlacel P135 by the company ICI.

As glycerol and/or sorbitan esters, mention may, for example, be made of polyglyceryl isostearate, such as the product sold under the name Isolan GI 34 by the company Goldschmidt; sorbitan isostearate, such as the product sold under the name Arlacel 987 by ICI; sorbitan glyceryl isostearate, such as the product sold under the name Arlacel 986 by the company ICI, and mixtures thereof.

For the O/W emulsions, mention may be made, for example, as emulsifiers, of non-ionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters such as the PEG-100 stearate/glyceryl stearate mixture sold, for example, by the company ICI under the name Arlacel 165; oxyalkylenated (oxyethylenated and/or oxypropylenated) fatty alcohol ethers; sugar esters such as sucrose stearate; ethers of a fatty alcohol and of a sugar, in particular alkylpolyglucosides (APGs), such as decylglucoside and laurylglucoside sold, for example, by the company Henkel under the respective names Plantaren 2000 and Plantaren 1200, cetostearyl glucoside optionally as a mixture with cetostearyl alcohol, sold, for example, under the name Montanov 68 by the company Seppic, under the name Tegocare CG90 by the company Goldschmidt and under the name Emulgade KE3302 by the company Henkel, and also arachidyl glucoside, for example in the form of the mixture of arachidyl and behenyl alcohols and arachidyl glucoside sold under the name Montanov 202 by the company Seppic. According to one particular embodiment of the invention, the mixture of alkylpolyglucoside as defined above with the corresponding fatty alcohol may be in the form of a self-emulsifying composition, as described, for example, in document WO-A-92/06778.

Among the other emulsion stabilisers, use will more particularly be made of polymers of isophthalic acid or of sulphoisophthalic acid, and in particular phthalate/sulphoisophthalate/glycol copolymers, for example the diethylene glycol/phthalate/isophthalate/1,4-cyclohexanedimethanol copolymer (INCI name: Polyester-5) sold under the names “Eastman AQ polymer” (AQ35S, AQ38S, AQ55S, AQ48 Ultra) by the company Eastman Chemical.

When an emulsion is involved, the aqueous phase of said emulsion may comprise a non-ionic vesicular dispersion prepared according to known methods (Bangham, Standish and Watkins. J. Mol. Biol. 13, 238 (1965), FR 2 315 991 and FR 2 416 008).

The compositions according to the invention find their use in a large number of treatments, in particular cosmetic treatments, for the skin, the lips and the hair, including the scalp, in particular for protecting and/or caring for the skin, the lips and/or the hair, and/or for making up the skin and/or the lips.

Another subject of the present invention is constituted of the use of the compositions according to the invention as defined above, for the manufacture of products for the cosmetic treatment of the skin, the lips, the nails, the hair, the eyelashes, the eyebrows and/or the scalp, in particular care products, sunscreen products and makeup products.

The cosmetic compositions according to the invention may, for example, be used as a makeup product.

The cosmetic compositions according to the invention may, for example, be used as a care product and/or sunscreen product for the face and/or the body, of liquid to semi-liquid consistency, such as milks, more or less rich creams, cream gels, or pastes. They may optionally be packaged in an aerosol and may be in the form of a foam or a spray.

The compositions according to the invention in the form of vaporisable fluid lotions in accordance with the invention are applied to the skin or the hair in the form of fine particles using pressurised devices. The devices in accordance with the invention are well known to those skilled in the art and comprise non-aerosol pump-dispensers or “atomisers”, aerosol containers comprising a propellant and also aerosol pump-dispensers that use compressed air as a propellant. The latter are described in U.S. Pat. No. 4,077,441 and U.S. Pat. No. 4,850,517 (forming an integral part of the content of the description).

The compositions packaged as an aerosol in accordance with the invention generally contain conventional propellants such as, for example, hydrofluoro compounds, dichlorodifluoromethane, difluoroethane, dimethyl ether, isobutane, n-butane, propane or trichlorofluoromethane. They are preferably present in amounts ranging from 15% to 50% by weight relative to the total weight of the composition.

Concrete, but in no way limiting, examples that illustrate the invention will now be given.

### Solubility of Merocyanins, Compared Between Prior Art Solvents and the Solvents of the Invention

### Screening Agents Tested:

### Screening Agent 1

{3-[(2-Ethylhexyl)amino]-5,5-dimethylcyclohex-2-en-1-ylidene}propanedinitrile

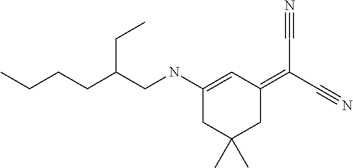

### Screening Agent 2

{(2E)-3-[bis(2-Ethylhexyl)amino]-prop-2-en-1-ylidene}propanedinitrile

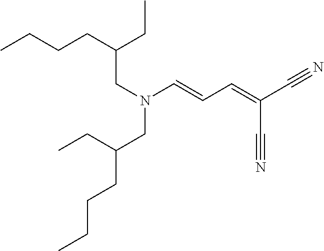

### Screening Agent 3

2-Ethylhexyl (2E,4E)-2-cyano-5-(diethylamino)penta-2,4-dienoate

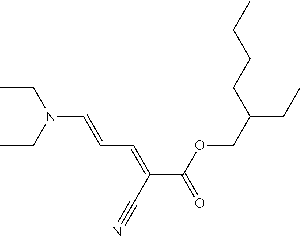

### Screening Agent 4

Octyl (2Z,4E)-5-(diethylamino)-2-(phenylsulphonyl)penta-2,4-dienoate

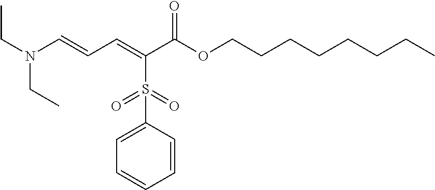

Screening agent 5
2-Ethylhexyl (2E,4E)-5-(diethyl-amino)-2-(phenylsulphonyl)penta-2,4-dienoate

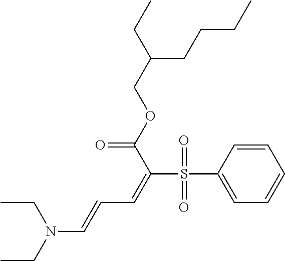

### Comparative Oil 1

Isopropyl N-lauroylsarcosinate (Eldew SL-205 from Ajinomoto) of formula:

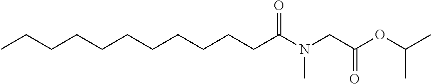

### Comparative Oil 2

Finsolv TN: C₁₂-C₁₅ alkyl benzoate

### Comparative Oil 3

Miglyol 812: Caprylic/capric acid triglycerides

### Comparative Oil 4

X-Tend: 2-Phenylethyl benzoate

### Procedure:

X mg of product are introduced into Y mg of oil; with mild heating (<60° C.) and the use of a sonicator for 1 minute, the solution obtained is left at laboratory temperature for 1 month; the state of this solution is observed; if no crystal or oily deposit is visible, the solubility of the product is considered to be greater than X×100/(X+Y) weight/weight; when crystals or an oily deposit appear, the test is repeated with 5% less product.

TABLE 1

| | Screening | Screening | Screening |
|---|---|---|---|
| Oil tested | agent 1 | agent 4 | agent 5 |
| Isopropyl N- | 33% | 35% | 50% gel |
| lauroyl | | | |
| sarcosinate | | | |
| C₁₂-C₁₅ alkyl | 16% | 20% | 50% gel |
| benzoate | | | |
| Caprylic/capric | 14% | 16% | 50% gel |
| acid triglycerides | | | |
| 2-Phenylethyl | 27% | 25% | 50% gel |
| benzoate | | | |
| Compound (m) of | 52% | 62% | 60% gel |
| the invention | | | |
| Compound (j) of | 53% | 52% | 60% gel |
| the invention | | | |
| Compound (l) of | 48% | 58% | 60% gel |
| the invention | | | |

TABLE 2

| | Screening |
|---|---|
| Oil tested | agent 3 |
| C₁₂-C₁₅ alkyl benzoate | 25% |
| Caprylic-capric acid | 22% |
| triglycerides | |
| 2-Phenylethyl benzoate | 32% |
| Compound (m) of the | 44% |
| invention | |
| Compound (j) of the | 47% |
| invention | |
| Compound (l) of the | 50% |
| invention | |

### FORMULATION EXAMPLES 1-4

The following formulations were prepared, the amounts are expressed as percentages by weight relative to the total weight of the composition.

| | | | | |
|---|---|---|---|---|
| Composition | Ex1 | Ex2 | Ex3 | Ex4 |

Phase A

| | | | | |
|---|---|---|---|---|
| Polydimethylsiloxane | 0.5 | 0.5 | 0.5 | 0.5 |
| Preservatives | 1 | 1 | 1 | 1 |
| Stearic acid | 1.5 | 1.5 | 1.5 | 1.5 |
| Glyceryl monostearate/PEG100 | 1 | 1 | 1 | 1 |
| stearate mixture | | | | |
| Mixture of cetylstearyl glucoside | 2 | 2 | 2 | 2 |
| and of cetyl, stearyl alcohols | | | | |
| Cetyl alcohol | 0.5 | 0.5 | 0.5 | 0.5 |
| Butyl 1-butyl-5-oxopyrrolidine-3- | 20 | | 15 | — |
| carboxylate (compound (m)) | | | | |
| Methyl 1-butyl-5-oxopyrrolidine-3- | | 20 | | 15 |
| carboxylate (compound (j)) | | | | |
| Compound (l) | 5 | 5 | | |
| Compound (14) | — | — | 3 | 3 |

Phase B

| | | | | |
|---|---|---|---|---|
| Deionised water | qs 100 | qs 100 | qs 100 | qs 100 |
| Glycerol | 5 | 5 | 5 | 5 |
| Xanthan gum | 0.2 | 0.2 | 0.2 | 0.2 |
| Monocetyl phosphate | 1 | 1 | 1 | 1 |

Phase C

| | | | | |
|---|---|---|---|---|
| Isohexadecane | 1 | 1 | 1 | 1 |
| Acrylic acid/stearyl methacrylate | 0.2 | 0.2 | 0.2 | 0.2 |
| copolymer | | | | |
| Triethanolamine | qs | qs | qs | qs |
| | pH | pH | pH | pH |

### Procedure:

The aqueous phase (Phase B) containing all its ingredients is heated to 80° C. in a waterbath. The fatty phase (Phase A) containing all its ingredients is heated to 80° C. in a waterbath. A is emulsified in B with rotor-stator stirring (instrument from the company Moritz). Phase C is incorporated and the resulting mixture is left to return to ambient temperature with moderate stirring. The triethanolamine is introduced so as to adjust the pH to the desired value at the end of production.

The anti-sun emulsions obtained are stable with respect to storage and do not display any crystals or precipitates.

1. Composition comprising, in a cosmetically acceptable medium, at least one ester derived from 4-carboxy-2-pyrrolidinone, of formula (I) below: 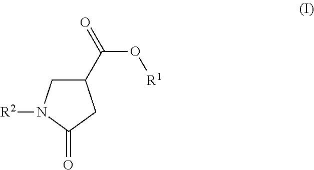
in which:
R¹ denotes a linear or branched C₁-C₂₀ alkyl radical,
R² denotes a linear or branched C₁-C₂₀ alkyl radical that may contain a C₅-C₆ ring, the phenyl radical, the benzyl radical or the phenethyl radical,
and at least one merocyanin-type UV screening agent.

2. Composition according to claim 1, in which the ester derived from 4-carboxy-2-pyrrolidinone, of formula (I), is chosen from the following compounds:

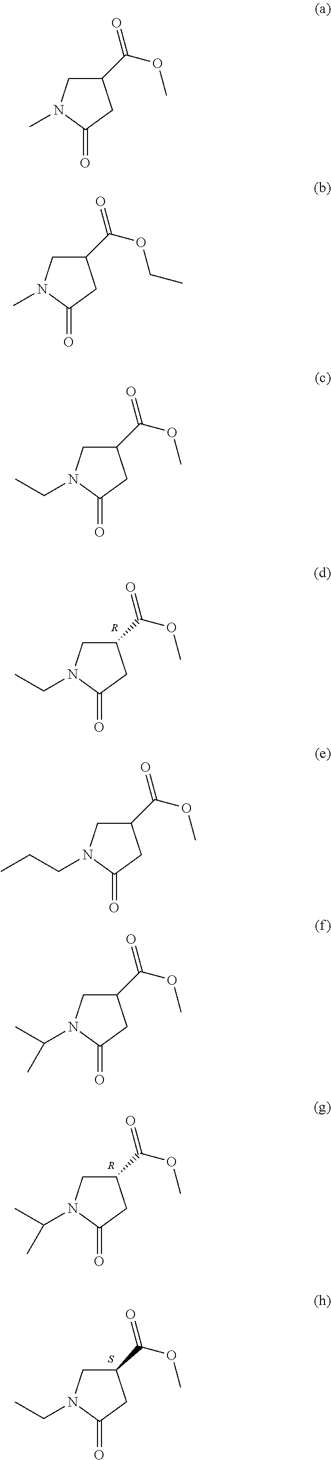

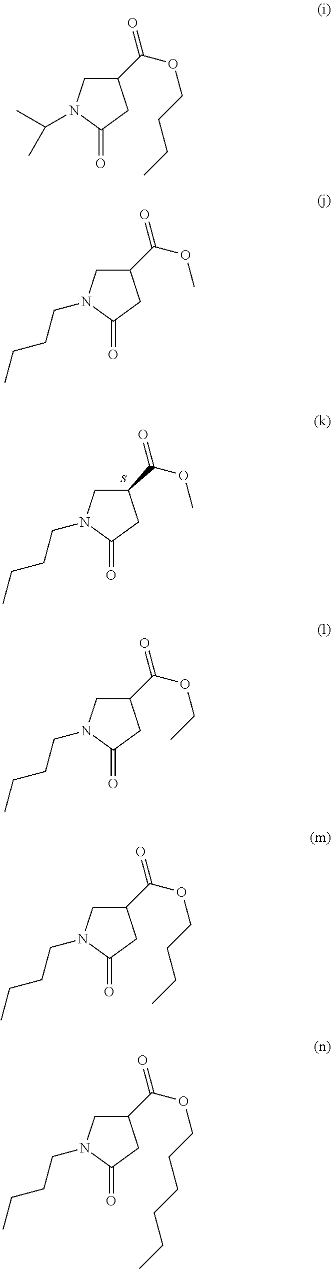

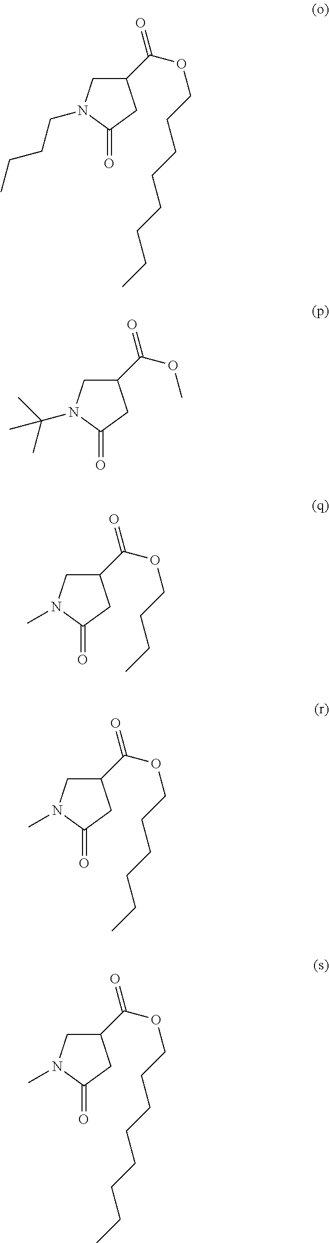

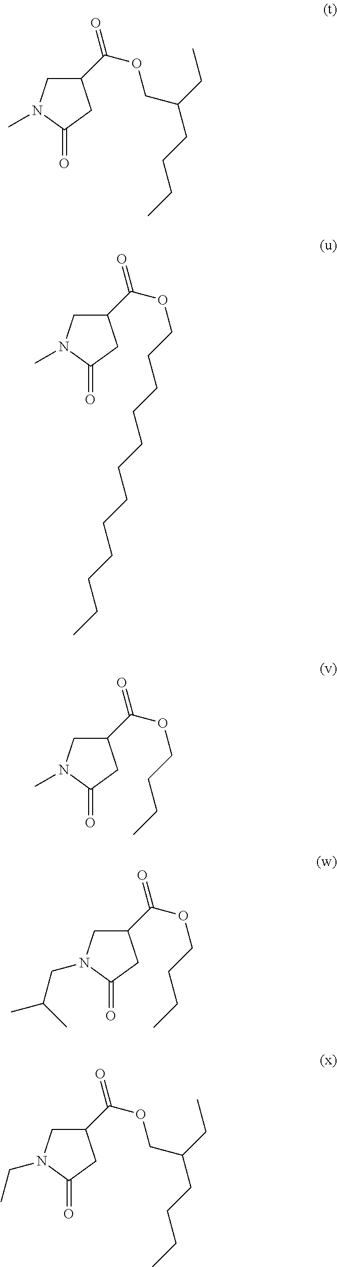

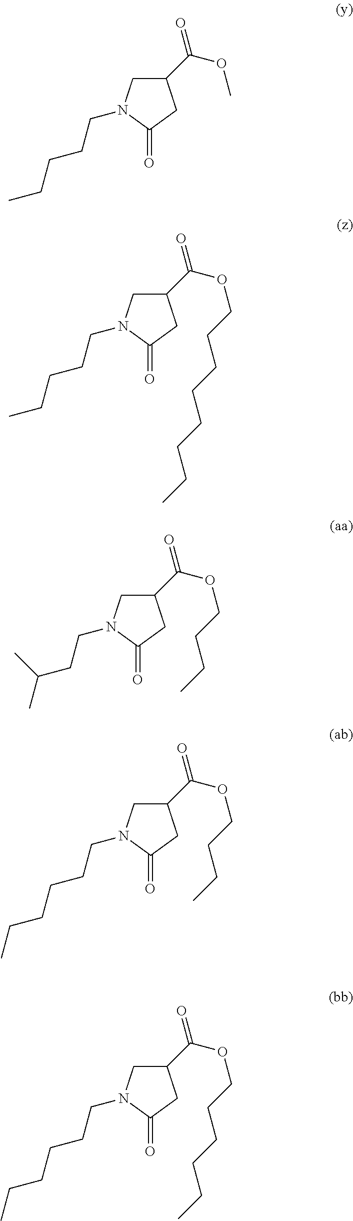

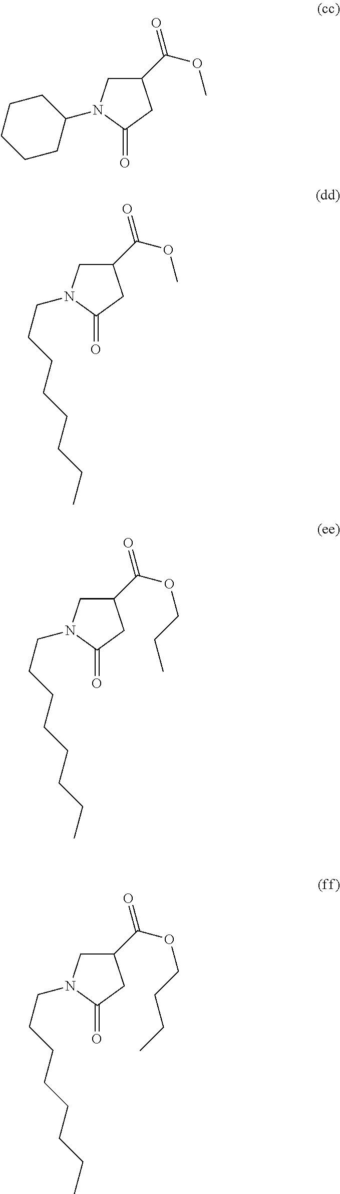

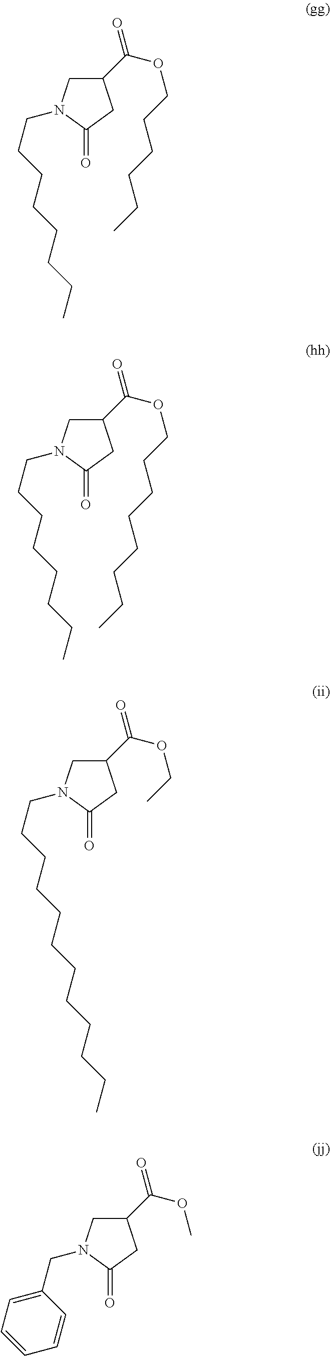

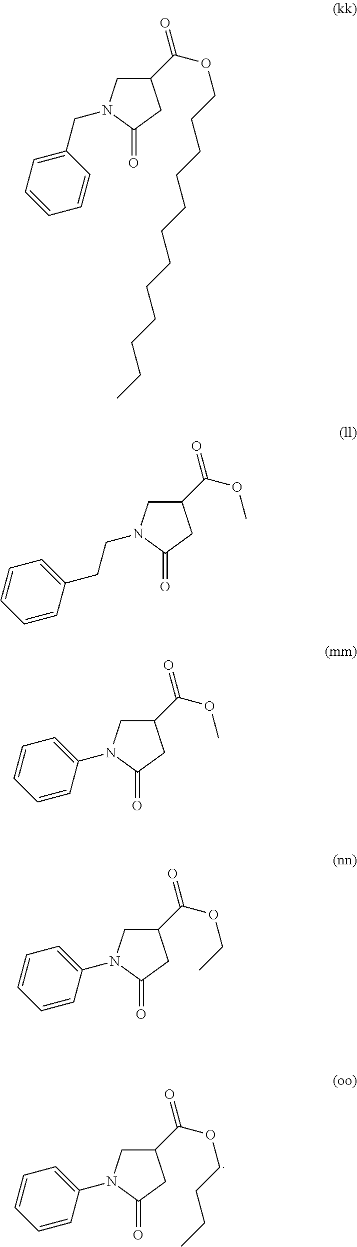

3. Composition according to claim 2, in which the compounds of formula (I) are chosen from the compounds (t), (u), (v), (x), (j), (l), (m), (w), (n), (ab), (ii) and (kk), and more particularly from the compounds (x), (j), (l), (m) and (ii).

4. Composition according to claim 1, in which the merocyanin UV screening agent is chosen from compounds (1) to (12) below: 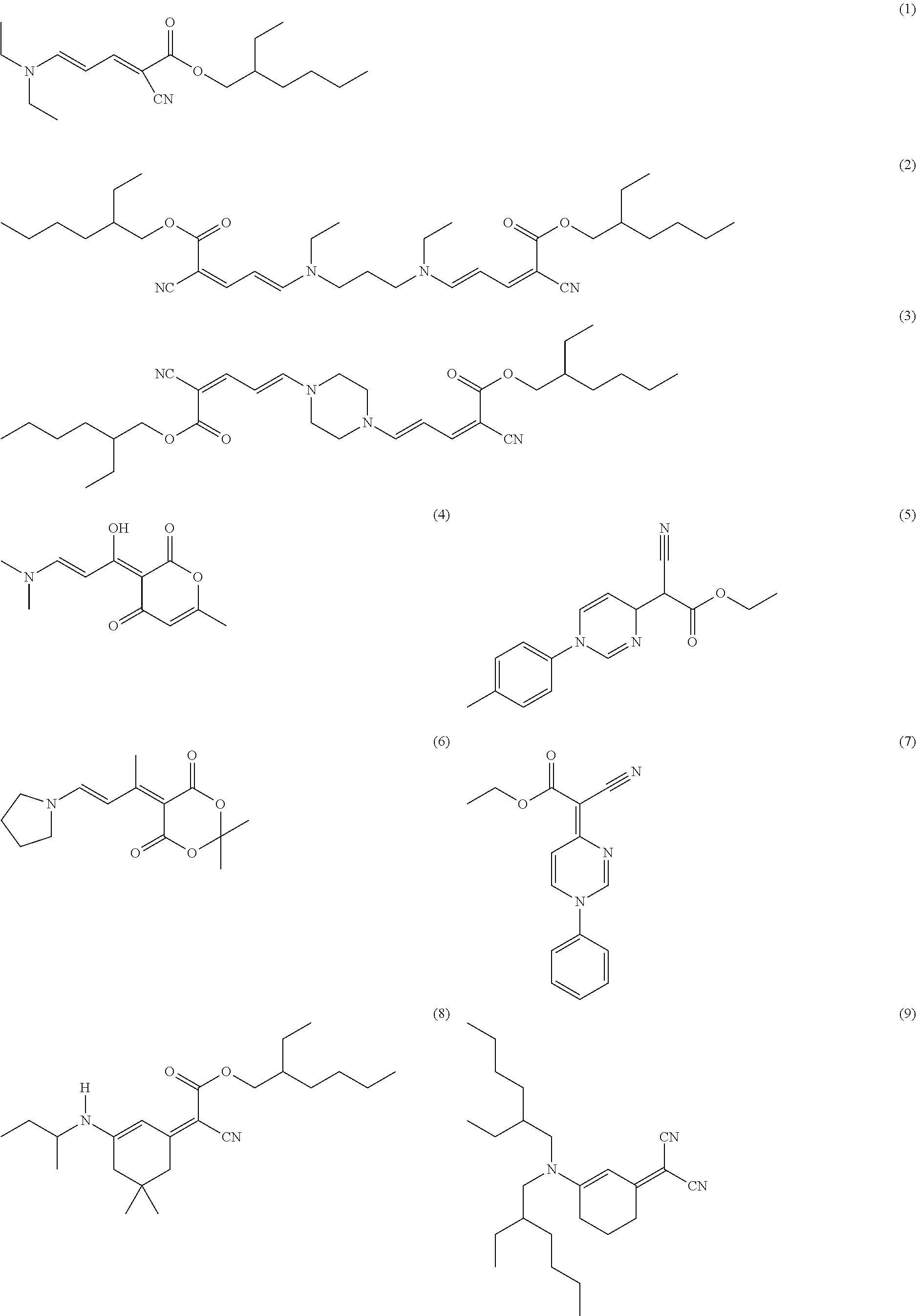
The compound {3-[(2-ethylhexyl)amino]-5,5-dimethylcyclohex-2-en-1-ylidene}propanedinitrile 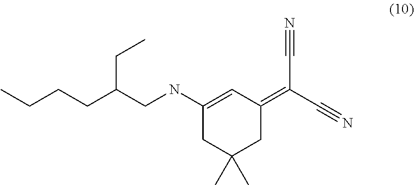
The compound {(2E)-3-[bis(2-ethylhexyl)amino]prop-2-en-1-ylidene}propanedinitrile 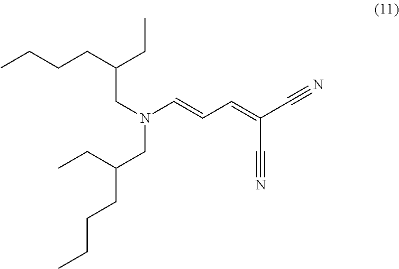
The compound 2-ethylhexyl(2E,4E)-2-cyano-5-(diethylamino)penta-2,4-dienoate

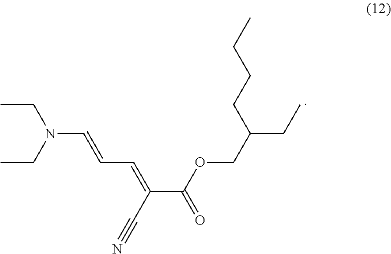

5. Composition according to claim 1, in which the merocyanin-type UV screening agent is chosen from the sulphone derivatives of merocyanin corresponding to one or other of formulae (VI) to (VIII) below: 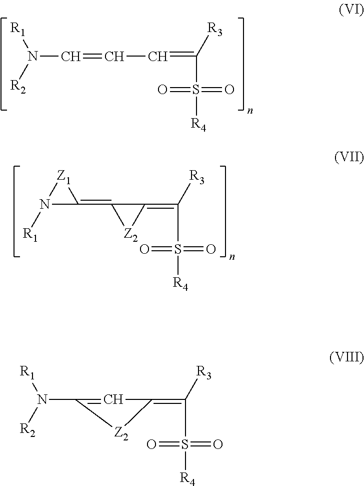
in which:
R₁ and R₂, which may be identical or different, represent H, a C₁-C₂₂ alkyl radical, a C₃-C₈ cycloalkyl radical or a C₆-C₂₀ aryl radical, in being understood that only one of R₁ and R₂ is H and that R₁ and R₂, together with the nitrogen, can form a ring containing the group —(CH₂)ₘ—, which is uninterrupted or interrupted with —O— or with —NH—,
R₃ represents a carboxyl, —COOR₅, —CONHR₅, —COR₅, —CONR₁R₅, —CN or SO₂R₅ group,
R₄ and R₅, which may be identical or different, represent a C₁-C₂₂ alkyl radical, a C₃-C₈ cycloalkyl radical or a C₆-C₂₀ aryl radical,
Z₁ and Z₂, which may be identical or different, are groups —(CH₂)₁—, uninterrupted or interrupted with —O—, with —S— or with —NR₆—, and/or unsubstituted or substituted with a C₁-C₆ alkyl radical,
R₆ is a C₁-C₅ alkyl radical,
l is 1-4,
m is 1-7,
n is 1-4,
with the proviso that
(i) when n=2, R₁, R₄ or R₅ is an alkyl diradical or R₁ and R₂, together with 2 nitrogen atoms, form a —(CH₂)ₘ— divalent radical,
(ii) when n=3, R₁, R₄ or R₅ is a trivalent radical,
(iii) when n=4, R₁, R₄ or R₅ is a tetravalent radical,
(iv) R₁ and R₂ are not simultaneously a hydrogen atom, and also the E,E-, E,Z- or Z,Z-isomeric forms thereof.

6. Composition according to claim 5, in which the merocyanin-type UV screening agent(s) is (are) chosen from the sulphone derivatives of merocyanin corresponding to formula (VI) and for which the following conditions are met:
R₁ and R₂, which may be identical or different, denote a C₁-C₁₂ alkyl;
R₃ denotes a COOR₅ group;
R₄ denotes a phenyl or tolyl group;
R₅ denotes C₁-C₁₂ alkyl;
n is equal to 1 or 2.

7. Composition according to claim 6, in which the compounds of formula (VI) are chosen from compounds (13) to (17) below:
Ethyl 5-(dihexylamino)-2-(phenylsulphonyl)-2,4-pentadienoate: 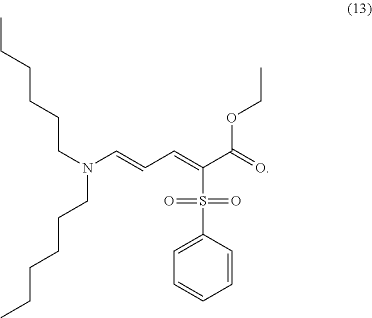
Octyl 5-N,N-diethylamino-2-phenylsulphonyl-2,4-pentadienoate: 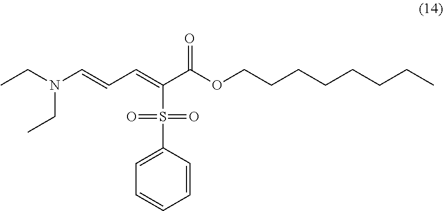
2-Ethylhexyl(2E,4E)-5-(diethylamino)-2-(phenylsulphonyl)penta-2,4-dienoate 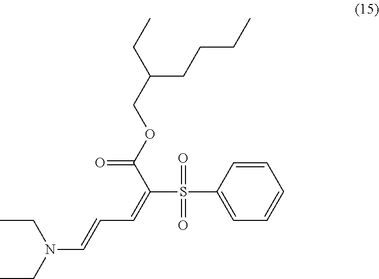
Lauryl 5-N,N-diethylamino-2-phenylsulphonyl-2,4-pentadienoate:

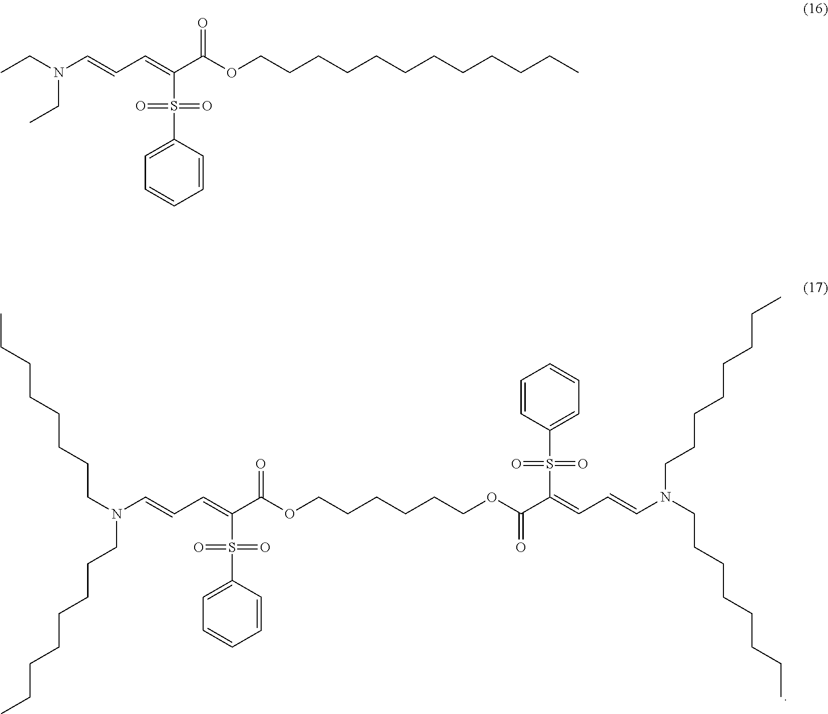

8. Composition according to claim 1, in which the merocyanin-type UV screening agent is chosen from the sulphone derivatives of merocyanin corresponding to one or other of formulae (IX) to (XI) below: 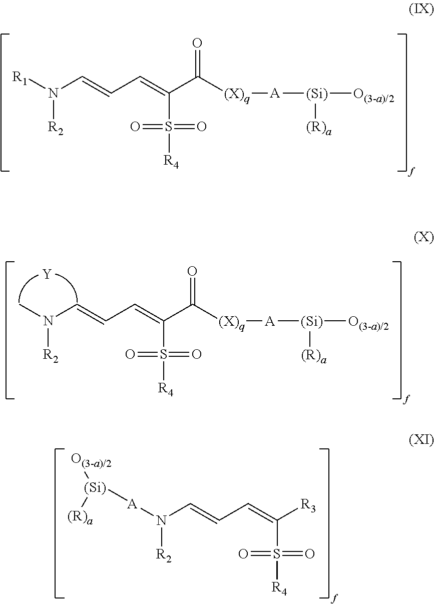
in which:
—X represents —O— or —NR₅—,
with R₁, R₂, R₃ and R₄ having the same definitions as in formulae (VI) (VII) and (VIII) indicated above,
o=0 or 1,
q=0 or 1,
Y is a divalent C₁-C₅ alkyl radical optionally substituted with C₁-C₄ alkyl radicals and/or containing —O—, —S— atoms, or with an —NR₁ group,
—R, which may be identical or different, represent a linear or branched, and optionally halogenated, C₁-C₂₀ alkyl radical, a C₆-C₁₂ aryl radical or a C₁-C₁₀ alkoxy group,
a=0 to 3,
A is a divalent radical chosen from methylene, ethylene or a group corresponding to one of formulae (XII), (XIII) or (XIV) below: 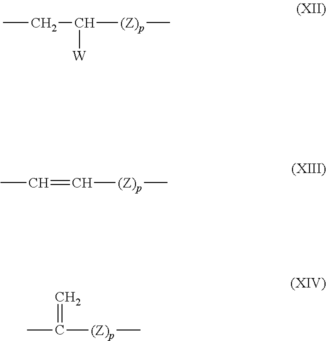
in which:
Z is a saturated or unsaturated, linear or branched C₁-C₆ alkylene radical optionally substituted with a hydroxyl radical or a saturated or unsaturated, linear or branched C₁-C₈ alkyl radical,
W represents a hydrogen atom, a hydroxyl radical or a saturated or unsaturated, linear or branched C₁-C₈ alkyl radical,
p is 0 or 1,
f=1 or 2; and also the E,E-, E,Z- or Z,Z-isomeric forms thereof.

9. Composition according to claim 8, in which formulae (IX) to (XI) comprise, in addition to the units of formula -A-(Si)(R)ₐ(O)_{(3-a)/2}, the organosiloxane may comprise units of formula (R)_{b}—(Si)(O)_{(4-b)/2} in which:
R has the same meaning as in formulae (VI) to (VIII),
b=1, 2 or 3.

10. Composition according to claim 8, in which the —(Si)(R)ₐ(O)_{(3-a)/2} groups may be represented by formula (XV), (XVI) or (XVII) below: 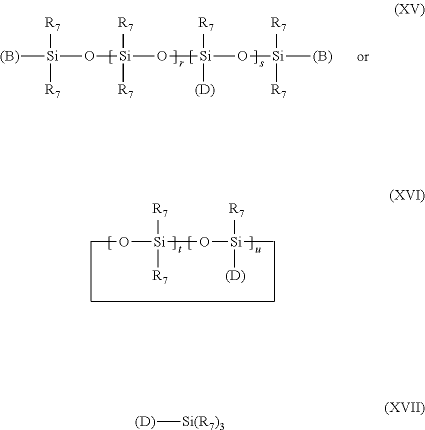
in which:
(D) is a linker between the silicone chain and the group A of the chromophores of formulae (IX) to (XI),
R₇, may be identical or different, are chosen from linear or branched C₁-C₃₀ alkyl radicals and phenyl, 3,3,3-trifluoropropyl and trimethylsilyloxy radicals, at least 80% by number of the R₆ radicals being methyl,
(B), which may be identical or different, are chosen from the radicals R₈ and the radical A,
r is an integer between 0 and 200, limits included, and s is an integer between 0 and 50, limits included, and if s=0, at least one of the two symbols (B) denotes A,
u is an integer between 1 and 10, limits included, and t is an integer between 0 and 10, limits included, it being understood that t+u is greater than or equal to 3.

11. Composition according to claim 8, in which the linear or cyclic diorganosiloxanes of formula (XV) or (XVI) are random polymers or oligomers having at least one, and even more preferably all, of the following characteristics:
R₈ is preferably methyl,
B is preferably methyl.

12. Composition according to claim 8, in which the merocyanin-type UV screening agent is chosen from the compounds of formulae (18) to (29) below:

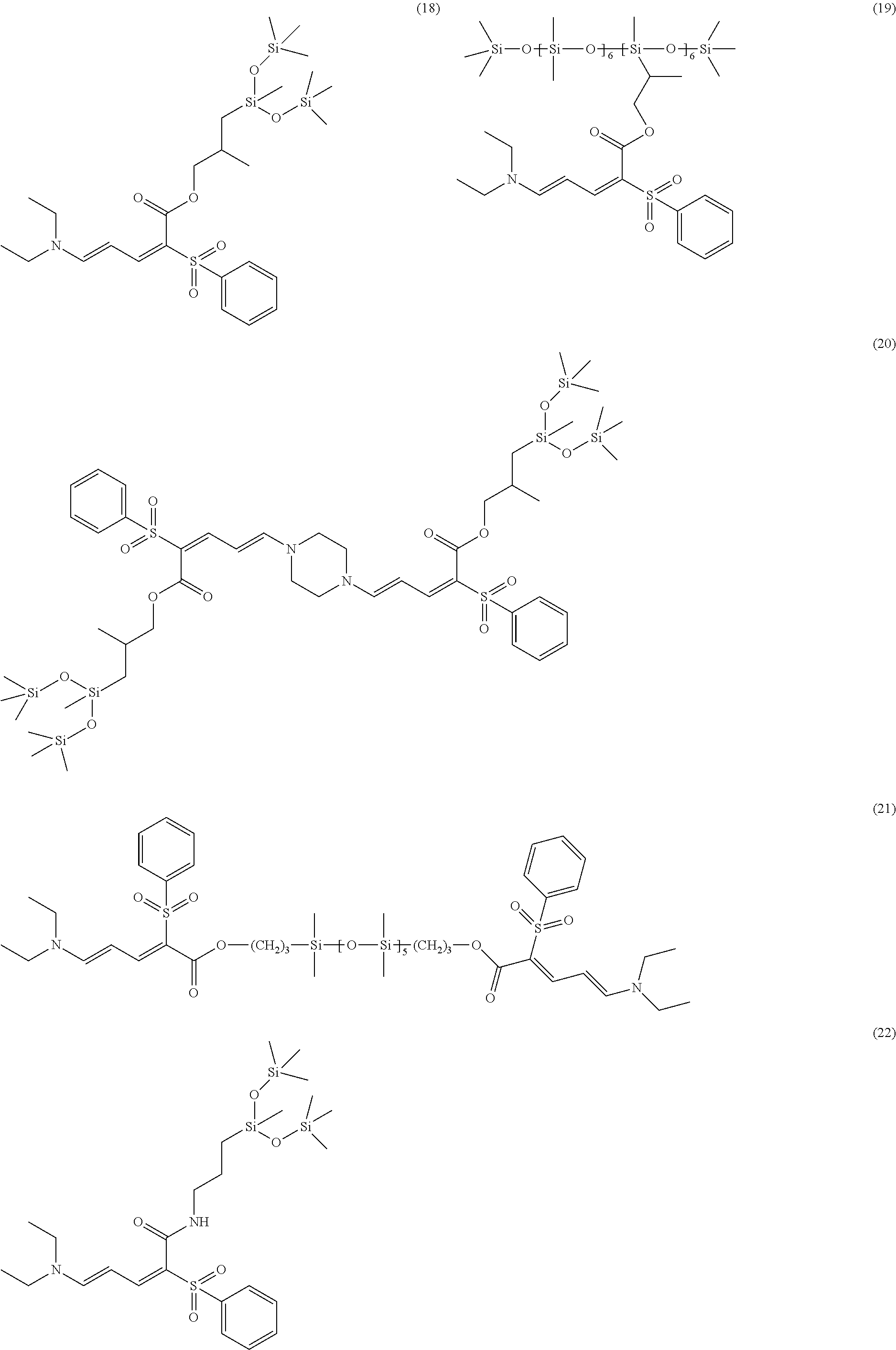

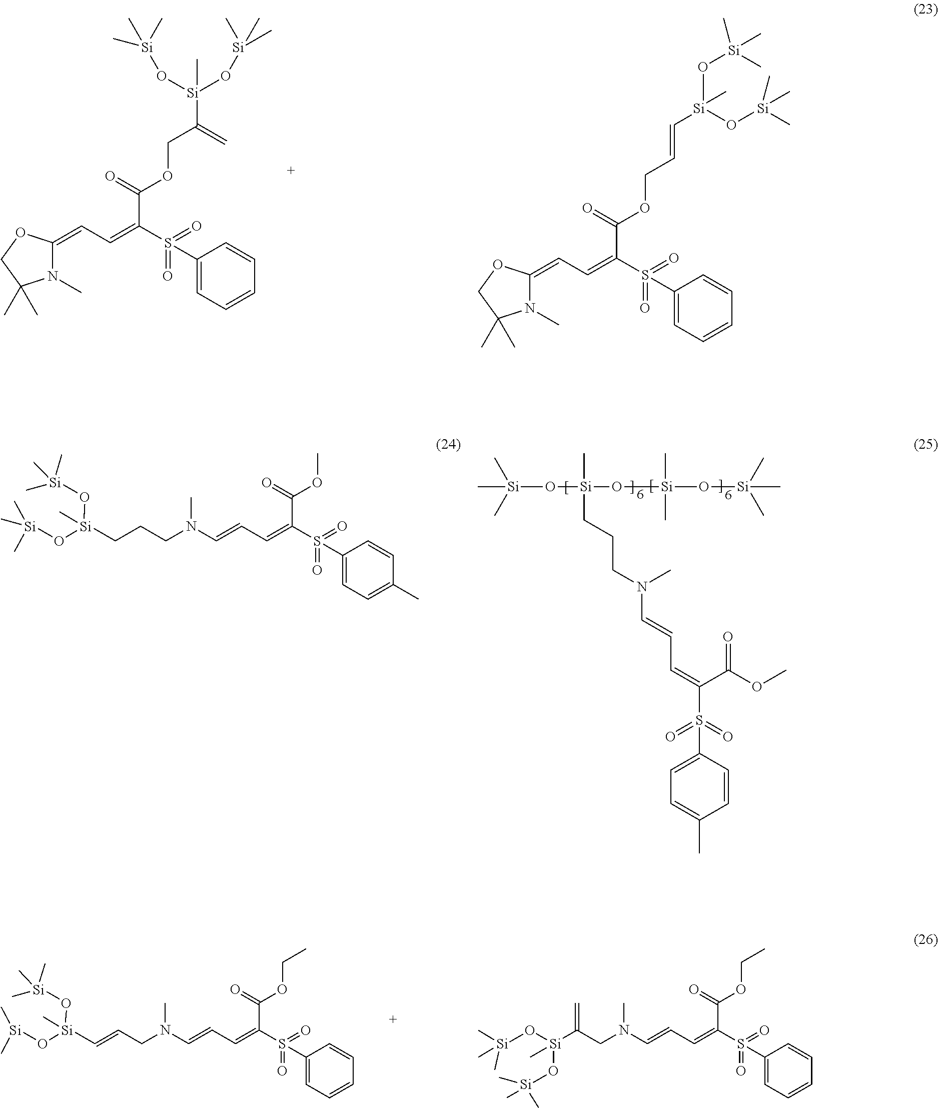

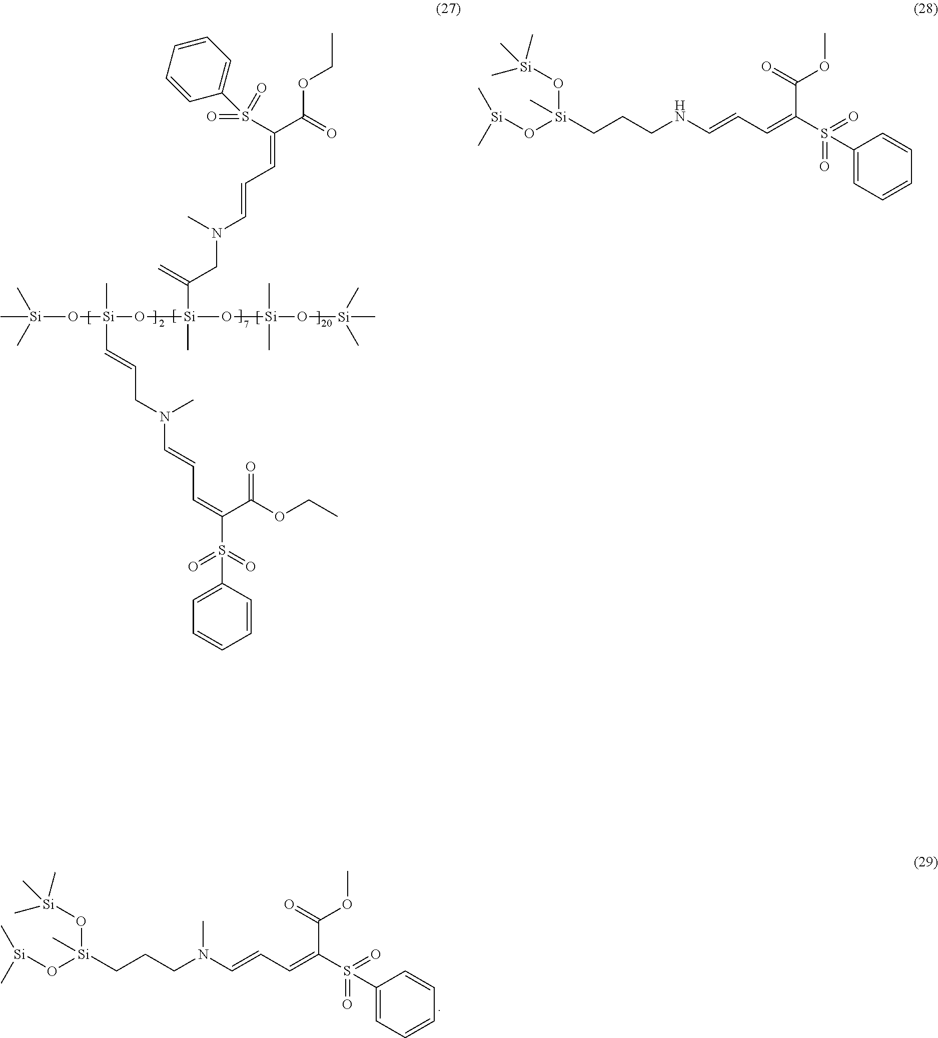

13. Composition according to claim 1, in which the derivative(s) of formula (I) is(are) present in an amount sufficient to solubilise, by itself or by themselves, the total amount of merocyanin screening agent(s) present in the composition.

14. Use of at least one ester derived from 4-carboxy-2-pyrrolidinone, of formula (I), as defined in claim 1, in a composition comprising, in a cosmetically acceptable medium, at least one merocyanin-type UV screening agent as defined in any one of the preceding claims, as a solvent for said merocyanin screening agent, in and/or as an agent for improving the solubility of said merocyanin screening agent in said composition.

15. Use of at least one ester derived from pyrrolidinone, of formula (I), as defined in claim 1, in a composition comprising, in a cosmetically acceptable medium, at least one merocyanin-type UV screening agent as defined in any one of the preceding claims, with the aim of improving the sun protection factor.

16. Composition according to claim 2, in which the merocyanin UV screening agent is chosen from compounds (1) to (12) below: 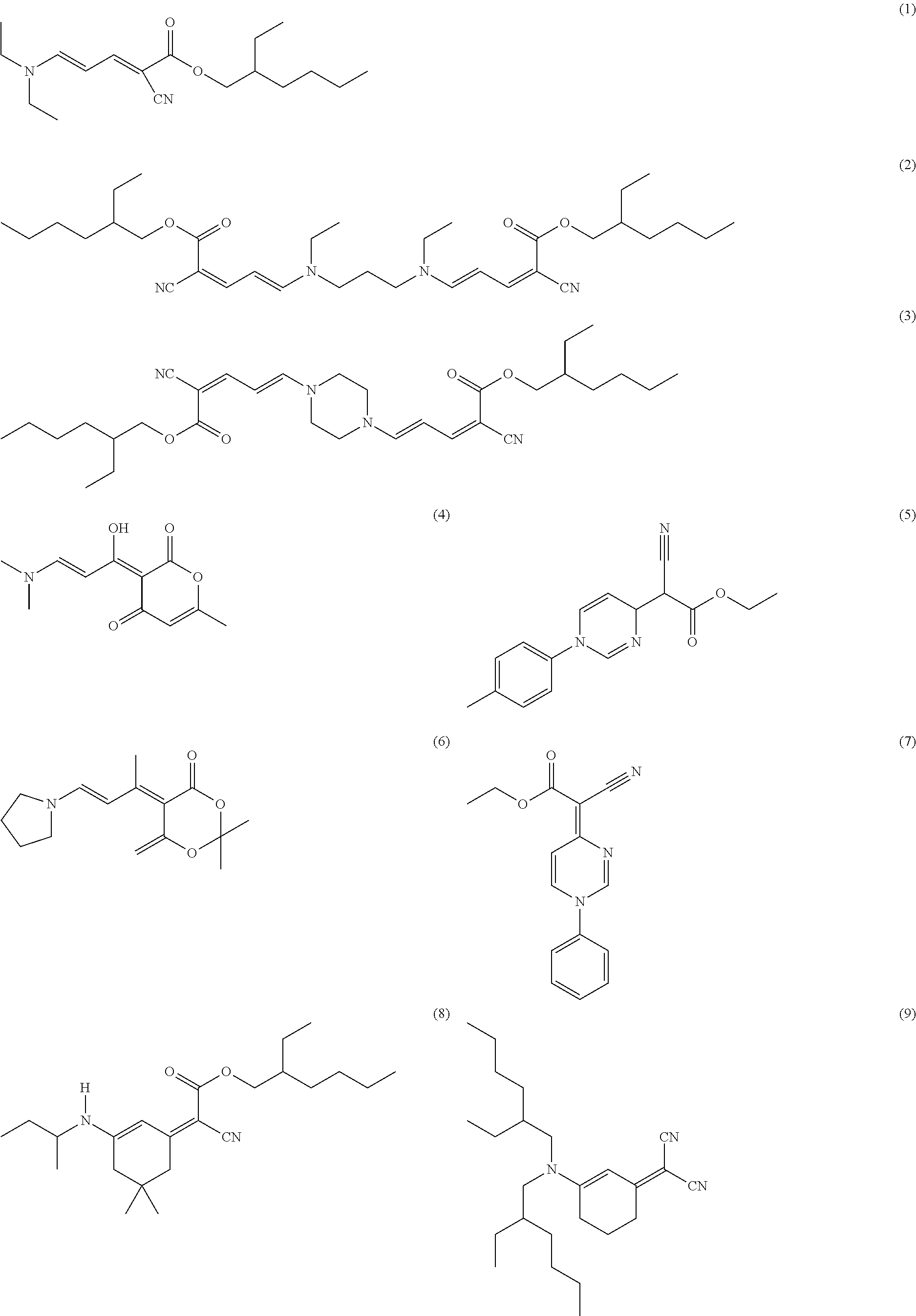
The compound {3-[(2-ethylhexyl)amino]-5,5-dimethylcyclohex-2-en-1-ylidene}propanedinitrile 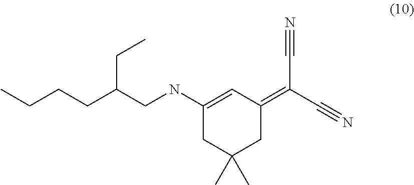
The compound {(2E)-3-[bis(2-ethylhexyl)amino]prop-2-en-1-ylidene}propanedinitrile 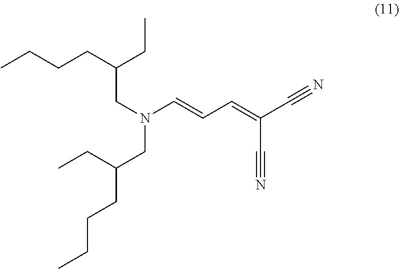
The compound 2-ethylhexyl(2E,4E)-2-cyano-5-(diethylamino)penta-2,4-dienoate

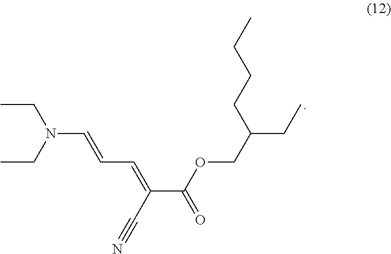

17. Composition according to claim 3, in which the merocyanin UV screening agent is chosen from compounds (1) to (12) below: 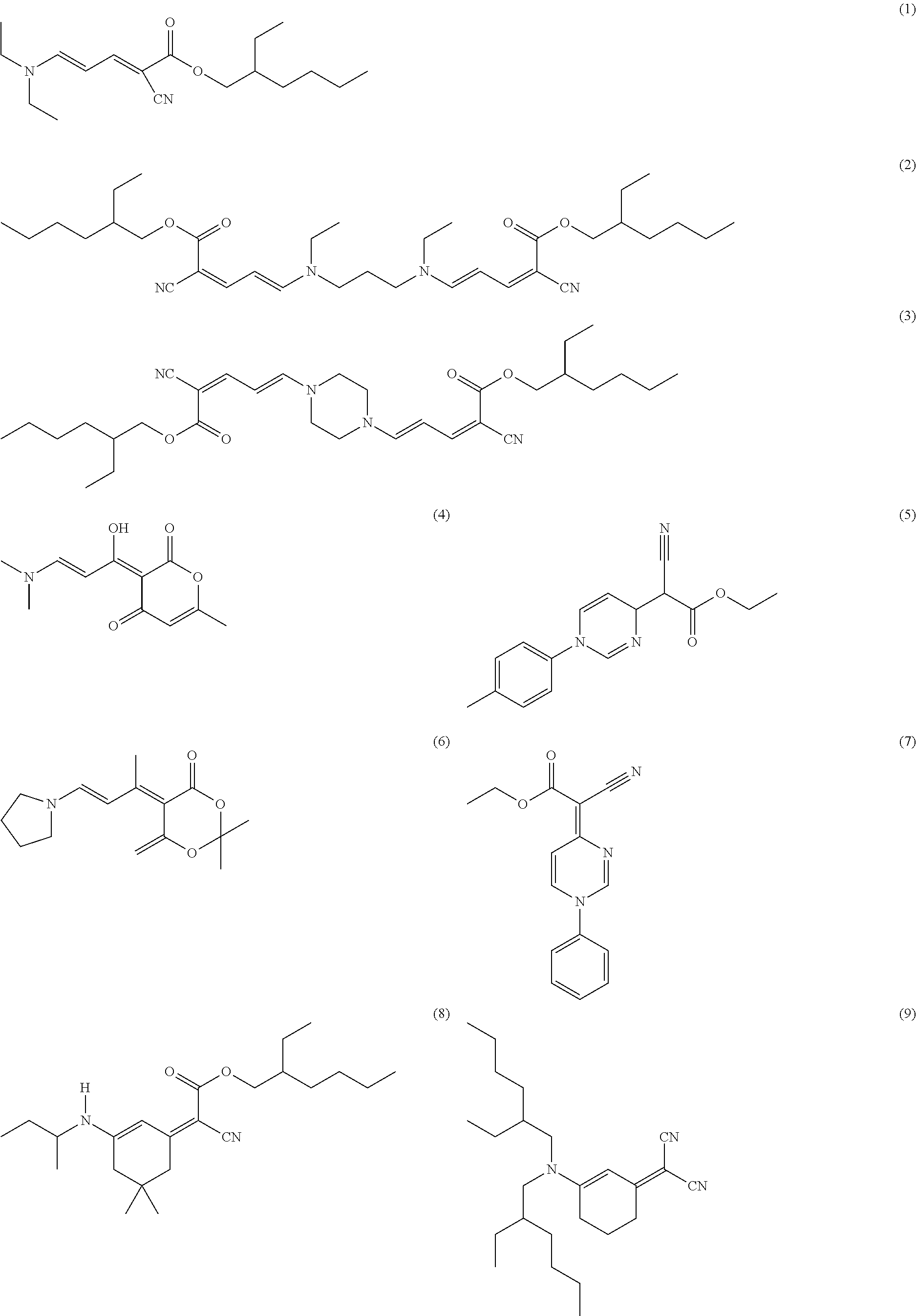
The compound {3-[(2-ethylhexyl)amino]-5,5-dimethylcyclohex-2-en-1-ylidene}propanedinitrile 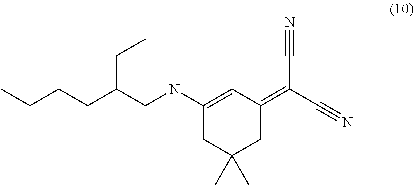
The compound {(2E)-3-[bis(2-ethylhexyl)amino]prop-2-en-1-ylidene}propanedinitrile 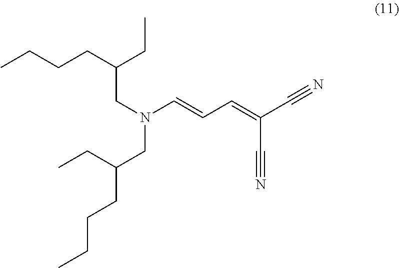
The compound 2-ethylhexyl(2E,4E)-2-cyano-5-(diethylamino)penta-2,4-dienoate

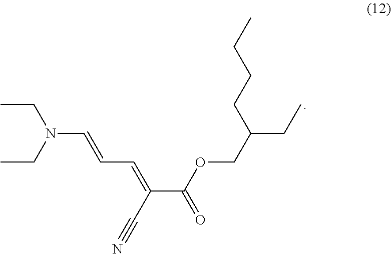

18. Composition according to claim 2, in which the merocyanin-type UV screening agent is chosen from the sulphone derivatives of merocyanin corresponding to one or other of formulae (VI) to (VIII) below: 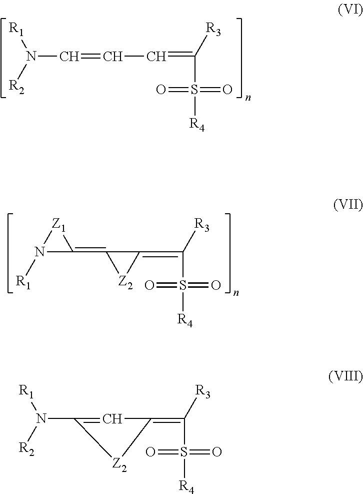
in which:
R₁ and R₂, which may be identical or different, represent H, a C₁-C₂₂ alkyl radical, a C₃-C₈ cycloalkyl radical or a C₆-C₂₀ aryl radical, in being understood that only one of R₁ and R₂ is H and that R₁ and R₂, together with the nitrogen, can form a ring containing the group —(CH₂)ₘ—, which is uninterrupted or interrupted with —O— or with —NH—,
R₃ represents a carboxyl, —COOR₅, —CONHR₅, —COR₅, —CONR₁R₅, —CN or SO₂R₅ group,
R₄ and R₅, which may be identical or different, represent a C₁-C₂₂ alkyl radical, a C₃-C₈ cycloalkyl radical or a C₆-C₂₀ aryl radical,
Z₁ and Z₂, which may be identical or different, are groups —(CH₂)₁—, uninterrupted or interrupted with —O—, with —S— or with —NR₆—, and/or unsubstituted or substituted with a C₁-C₆ alkyl radical,
R₆ is a C₁-C₅ alkyl radical,
l is 1-4,
m is 1-7,
n is 1-4,
with the proviso that
(i) when n=2, R₁, R₄ or R₅ is an alkyl diradical or R₁ and R₂, together with 2 nitrogen atoms, form a —(CH₂)ₘ— divalent radical,
(ii) when n=3, R₁, R₄ or R₅ is a trivalent radical,
(iii) when n=4, R₁, R₄ or R₅ is a tetravalent radical,
(iv) R₁ and R₂ are not simultaneously a hydrogen atom, and also the E,E-, E,Z- or Z,Z-isomeric forms thereof.

19. Composition according to claim 3, in which the merocyanin-type UV screening agent is chosen from the sulphone derivatives of merocyanin corresponding to one or other of formulae (VI) to (VIII) below: 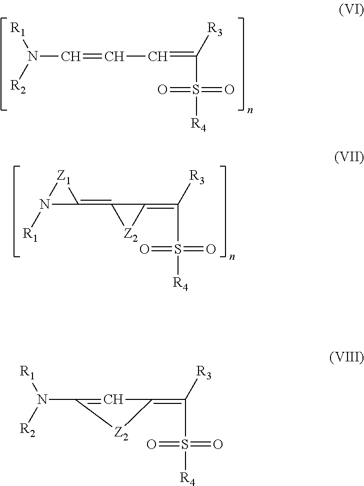
in which:
R₁ and R₂, which may be identical or different, represent H, a C₁-C₂₂ alkyl radical, a C₃-C₈ cycloalkyl radical or a C₆-C₂₀ aryl radical, in being understood that only one of R₁ and R₂ is H and that R₁ and R₂, together with the nitrogen, can form a ring containing the group —(CH₂)ₘ—, which is uninterrupted or interrupted with —O— or with —NH—,
R₃ represents a carboxyl, —COOR₅, —CONHR₅, —COR₅, —CONR₁R₅, —CN or SO₂R₅ group,
R₄ and R₅, which may be identical or different, represent a C₁-C₂₂ alkyl radical, a C₃-C₈ cycloalkyl radical or a C₆-C₂₀ aryl radical,
Z₁ and Z₂, which may be identical or different, are groups —(CH₂)₁—, uninterrupted or interrupted with —O—, with —S— or with —NR₆—, and/or unsubstituted or substituted with a C₁-C₆ alkyl radical,
R₆ is a C₁-C₅ alkyl radical,
l is 1-4,
m is 1-7,
n is 1-4,
with the proviso that
(i) when n=2, R₁, R₄ or R₅ is an alkyl diradical or R₁ and R₂, together with 2 nitrogen atoms, form a —(CH₂)ₘ— divalent radical,
(ii) when n=3, R₁, R₄ or R₅ is a trivalent radical,
(iii) when n=4, R₁, R₄ or R₅ is a tetravalent radical,
(iv) R₁ and R₂ are not simultaneously a hydrogen atom, and also the E,E-, E,Z- or Z,Z-isomeric forms thereof.

20. Composition according to claim 2, in which the merocyanin-type UV screening agent is chosen from the sulphone derivatives of merocyanin corresponding to one or other of formulae (IX) to (XI) below: 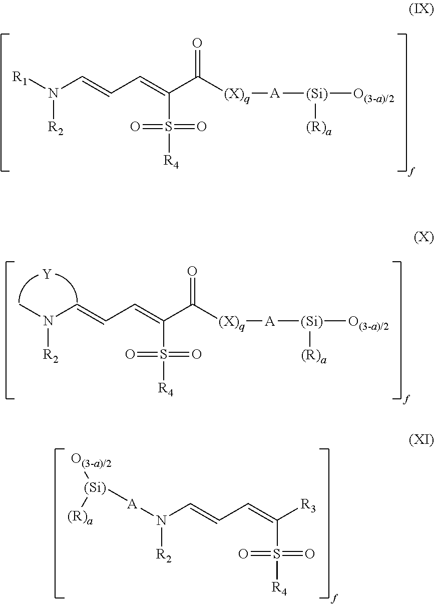
in which:
X represents —O— or —NR₅—,
with R₁, R₂, R₃ and R₄ having the same definitions as in formulae (VI) (VII) and (VIII) indicated above,
o=0 or 1,
q=0 or 1,
Y is a divalent C₁-C₅ alkyl radical optionally substituted with C₁-C₄ alkyl radicals and/or containing —O—, —S— atoms, or with an —NR₁ group,
R, which may be identical or different, represent a linear or branched, and optionally halogenated, C₁-C₂₀ alkyl radical, a C₆-C₁₂ aryl radical or a C₁-C₁₀ alkoxy group,
a=0 to 3,
A is a divalent radical chosen from methylene, ethylene or a group corresponding to one of formulae (XII), (XIII) or (XIV) below: 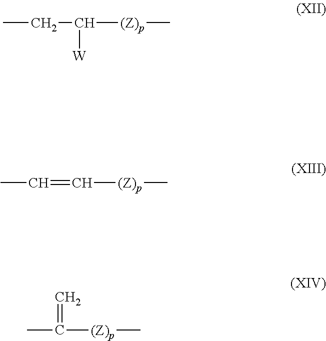
in which:
Z is a saturated or unsaturated, linear or branched C₁-C₆ alkylene radical optionally substituted with a hydroxyl radical or a saturated or unsaturated, linear or branched C₁-C₈ alkyl radical,
W represents a hydrogen atom, a hydroxyl radical or a saturated or unsaturated, linear or branched C₁-C₈ alkyl radical,
p is 0 or 1,
f=1 or 2; and also the E,E-, E,Z- or Z,Z-isomeric forms thereof.
